(19) United States
(12) Patent Application Publication
(10) Pub. No.: US 2021/0112743 A1
(43) Pub. Date: Apr. 22, 2021

(54) METHODS OF PRODUCING CBG-DOMINANT CANNABIS VARIETIES

(71) Applicant: BIOMAX SEEDS S.L.U., Valencia (ES)

(72) Inventors: Ernesto Rafael Llosá Llácer, Valencia (ES); Salvador Soler Aleixandre, Valencia (ES)

(73) Assignee: BIOMAX SEEDS S.L.U., Valencia (ES)

(21) Appl. No.: 17/076,036

(22) Filed: Oct. 21, 2020

Related U.S. Application Data
(63) Continuation-in-part of application No. 16/602,514, filed on Oct. 22, 2019, now Pat. No. PP32725.

(30) Foreign Application Priority Data
Oct. 31, 2019 (QZ) 2019/2758
Nov. 21, 2019 (XU) 202000033
Jan. 6, 2020 (XU) 202000032

(51) Int. Cl. A01H 6/28; A01H 5/12

(52) U.S. Cl. CPC A01H 6/28; A01H 5/12

(57) ABSTRACT

Cannabis plants named ‘PAN2020’, ‘HURV19PAN’, and ‘HURV2019CKH’ are disclosed. Compositions and methods for the breeding, production, processing, and use of Cannabis plants comprising a cannabinoid profile of greater than 10% CBG and less than 0.3%, 0.1%, or 0.05% of THC and CBD are disclosed. Embodiments include seeds of ‘PAN2020’, ‘HURV19PAN’, and/or ‘HURV2019CKH’, plants of ‘PAN2020’, ‘HURV19PAN’, and/or ‘HURV2019CKH’, plant parts thereof, methods for producing a Cannabis plant by crossing ‘PAN2020’, ‘HURV19PAN’, and/or ‘HURV2019CKH’ with itself or with another Cannabis plant variety, and methods for producing other Cannabis plant lines or plant parts derived from ‘PAN2020’, ‘HURV19PAN’, and/or ‘HURV2019CKH’, and the Cannabis plants, varieties, and their parts derived from the use of those methods. Also disclosed are Cannabis varieties, breeding varieties, plant parts, and cells derived from Cannabis plant ‘PAN2020’, ‘HURV19PAN’, and/or ‘HURV2019CKH’ are disclosed.

### CROSS-REFERENCE TO RELATED APPLICATIONS

This application is a continuation-in-part application of U.S. Plant Patent application Ser. No. 16/602,514, filed on Oct. 22, 2019, and claims priority to Plant Variety Protection Application No. 202000032, filed on Jan. 6, 2020, priority to Plant Variety Protection Application No. 202000033, filed on Nov. 21, 2019, and priority to Plant Breeders' Right Application Number 2019/2758, which was filed at Community Plant Variety Office in the European Union on Oct. 31, 2019, the disclosures of which are incorporated herein by reference in their entirety.

### BACKGROUND

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. The foregoing examples of the related art and limitations related therewith are intended to be illustrative and not exclusive. Moreover, it should be understood that after reading the teachings of the present invention, those skilled in the art can make various changes or modification to the present invention, and these equivalent forms also fall within the scope defined by the claims of the present application.

Cannabis sativa (L.) (Cannabis plant) is a species of flowering plants in the family Cannabaceae. Cannabis plants can be propagated from many means including seed, cuttings, and tissue culture. Seed, cuttings, and tissue culture germination protocols for Cannabis plants are well-known in the art.

Cannabis has many medical and therapeutic uses. For example, Cannabis has successfully been used to help relieve nausea and vomiting in patients undergoing chemotherapy treatment. Cannabis also has efficacy as an antiemetic as compared to other currently available pharmaceutical products.

Cannabis sativa (L.) contains several chemical compounds that are part of the cannabinoid family. Namely, the following five compounds can be found in Cannabis sativa (L.): cannabidiol (CBD), cannabichromene, cannabigerol (CBG), Δ-9-tetrahydrocannabinol (THC), and cannabinol. Cannabinoids from C. sativa (L.) are known for their antibacterial potential as well as other useful properties. CBG is of particular interest as an extract of Cannabis sativa (L.). However, all currently known species of Cannabis plant contain relatively low levels of CBG, e.g., 1.9% or less CBG by weight.

CBG is the non-acidic form of cannabigerolic acid, the parent molecule from which other cannabinoids are synthesized. CBG has the following chemical formula:

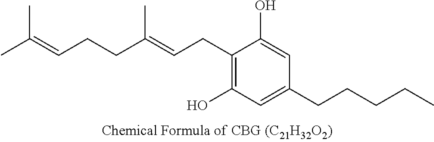 [SMILES: CCCCCc1cc(O)c(C/C=C(\C)CCC=C(C)C)c(O)c1]

Cannabis plants are an important and valuable plant, especially for medical and therapeutic uses. Thus, a continuing goal of Cannabis plant breeders is to develop plants with novel cannabinoid profiles. There is a long-felt need for Cannabis plants with a high content of CBG and low content/void of CBD and/or THC. The inventions described herein meet this long-felt need.

### SUMMARY

It is to be understood that this invention is not limited to particular embodiments described, as such may, of course, vary. The following embodiments and aspects thereof are described in conjunction with systems, tools, and methods which are meant to be exemplary, not limiting in scope.

According to one embodiment, there is provided new and distinct varieties of Cannabis plant, named ‘PAN2020’, ‘HURV19PAN’, and ‘HURV2019CKH’, characterized by their unique cannabinoid profile, specifically with respect to high cannabigerol (CBG) content (10% or higher) and lack of tetrahydrocannabinol (THC) and cannabidol (CBD).

According to one embodiment, there is provided a Cannabis plant ‘PAN2020’ which is valued as breeding line enabling the development of superior Cannabis plants. According to one embodiment, there is provided a Cannabis plant ‘HURV19PAN’ which is valued as breeding line enabling the development of superior Cannabis plants. According to one embodiment, there is provided a Cannabis plant ‘HURV2019CKH’ which is valued as breeding line enabling the development of superior Cannabis plants.

Another embodiment discloses a Cannabis plant ‘PAN2020’, wherein a representative sample of plant tissue of said Cannabis plant is to be deposited. Another embodiment discloses a Cannabis plant ‘HURV19PAN’, wherein a representative sample of seed producing said Cannabis plant is to be deposited. Another embodiment discloses a Cannabis plant ‘HURV2019CKH’, wherein a representative sample of plant tissue of said Cannabis plant is to be deposited.

Another embodiment discloses a Cannabis plant having all of the physiological and morphological characteristics of a Cannabis plant of variety ‘PAN2020’. Another embodiment discloses a Cannabis plant having all of the physiological and morphological characteristics of a Cannabis plant of variety ‘HURV19PAN’. Another embodiment discloses a Cannabis plant having all of the physiological and morphological characteristics of a Cannabis plant of variety ‘HURV2019CKH’.

Another embodiment relates to a method of producing a Cannabis plant, said method comprising cultivating a plant part comprising at least one cell of the Cannabis plant variety ‘PAN2020’. Another embodiment relates to a method of producing a Cannabis plant, said method comprising cultivating a plant part comprising at least one cell of the Cannabis plant variety ‘HURV19PAN’. Another embodiment relates to a method of producing a Cannabis plant, said method comprising cultivating a plant part comprising at least one cell of the Cannabis plant variety ‘HURV2019CKH’.

Another embodiment relates to tissue or cell culture of regenerable cells produced from a Cannabis plant of variety ‘PAN2020’. A further embodiment relates to a Cannabis plant regenerated from the tissue or cell culture of ‘PAN2020’. Another embodiment relates to tissue or cell culture of regenerable cells produced from a Cannabis plant of variety ‘HURV19PAN’. A further embodiment relates to a Cannabis plant regenerated from the tissue or cell culture of ‘HURV19PAN’. Another embodiment relates to tissue or cell culture of regenerable cells produced from a Cannabis plant of variety ‘HURV2019CKH’. A further embodiment relates to a Cannabis plant regenerated from the tissue or cell culture of ‘HURV2019CKH’.

Another embodiment relates to tissue or cell culture produced from tissues, protoplasts, or cells from the Cannabis plants disclosed in the subject application. In further embodiments, said tissues, cells, or protoplasts are produced from a plant part selected from the group consisting of pollen, embryos, protoplasts, meristematic cells, callus, pollen, leaves, ovules, anthers, cotyledons, hypocotyl, pistils, roots, root tips, flowers, seeds, petiole, and stems.

Another embodiment relates to a method of vegetatively propagating the plant of variety ‘PAN2020’, comprising the steps of: collecting tissue or cells capable of being propagated from a plant of ‘PAN2020’; cultivating said tissue or cells to obtain proliferated shoots; and rooting said proliferated shoots to obtain rooted plantlets; or cultivating said tissue or cells to obtain proliferated shoots, or to obtain plantlets and a plant produced by growing the plantlets or proliferated shoots of said plant. Another embodiment relates to a method of vegetatively propagating the plant of variety ‘HURV19PAN’, comprising the steps of: collecting tissue or cells capable of being propagated from a plant of ‘HURV19PAN’; cultivating said tissue or cells to obtain proliferated shoots; and rooting said proliferated shoots to obtain rooted plantlets; or cultivating said tissue or cells to obtain proliferated shoots, or to obtain plantlets and a plant produced by growing the plantlets or proliferated shoots of said plant. Another embodiment relates to a method of vegetatively propagating the plant of variety ‘HURV2019CKH’, comprising the steps of: collecting tissue or cells capable of being propagated from a plant of ‘HURV2019CKH’; cultivating said tissue or cells to obtain proliferated shoots; and rooting said proliferated shoots to obtain rooted plantlets; or cultivating said tissue or cells to obtain proliferated shoots, or to obtain plantlets and a plant produced by growing the plantlets or proliferated shoots of said plant.

Another embodiment relates to methods of developing a Cannabis plant variety having the physiological and morphological characteristics of a Cannabis plant of variety ‘PAN2020’, said method comprising genotyping a Cannabis plant of variety ‘PAN2020’, wherein said genotyping comprises obtaining a sample of nucleic acids from said plant and detecting in said nucleic acids a plurality of polymorphisms, and using said identified polymorphisms for marker assisted selection in a breeding program. Another embodiment relates to methods of developing a Cannabis plant variety having the physiological and morphological characteristics of a Cannabis plant of variety ‘HURV19PAN’, said method comprising genotyping a Cannabis plant of variety ‘HURV19PAN’, wherein said genotyping comprises obtaining a sample of nucleic acids from said plant and detecting in said nucleic acids a plurality of polymorphisms, and using said identified polymorphisms for marker assisted selection in a breeding program. Another embodiment relates to methods of developing a Cannabis plant variety having the physiological and morphological characteristics of a Cannabis plant of variety ‘HURV2019CKH’, said method comprising genotyping a Cannabis plant of variety ‘HURV2019CKH’, wherein said genotyping comprises obtaining a sample of nucleic acids from said plant and detecting in said nucleic acids a plurality of polymorphisms, and using said identified polymorphisms for marker assisted selection in a breeding program.

Another embodiment relates to a method for developing a Cannabis plant variety, comprising identifying and selecting a spontaneous mutation of a Cannabis plant selected from the varieties consisting of ‘PAN2020’, ‘HURV19PAN’, and ‘HURV2019CKH’ or a part thereof, and cultivating said selected spontaneous mutation plant or plant part. A further embodiment relates to a Cannabis plant produced by cultivating said selected spontaneous mutation plant or plant part.

Another embodiment relates to a method for developing a Cannabis plant variety, comprising introducing a mutation into the genome of a plant selected from the varieties consisting of ‘PAN2020’, ‘HURV19PAN’, and ‘HURV2019CKH’ or a part thereof, and cultivating said mutated plant or plant part. A further embodiment relates to a Cannabis plant produced by cultivating said mutated plant or plant part. In a further embodiment, said mutation is introduced using a method such as temperature, long-term seed storage, tissue culture conditions, ionizing radiation, chemical mutagens, targeting induced local lesions in genomes, zinc finger nuclease mediated mutagenesis, CRISPR/Cas-9, meganucleases, or gene editing.

Another embodiment relates to a method for developing a Cannabis plant variety, comprising transforming a Cannabis plant selected from the varieties consisting of ‘PAN2020’, ‘HURV19PAN’, and ‘HURV2019CKH’, with a transgene. In a further embodiment, said transgene confers resistance to an herbicide, insecticide, or disease. A further embodiment relates to an herbicide, insecticide, or disease resistant plant produced by the method for developing a Cannabis plant variety, comprising transforming a Cannabis plant selected from the varieties consisting of ‘PAN2020’, ‘HURV19PAN’, and ‘HURV2019CKH’, with a transgene.

Another embodiment relates to a method of producing an F₁ seed or embryo, wherein the method comprises crossing a Cannabis plant of variety ‘PAN2020’ with a second plant and harvesting the resultant F₁ seed or embryo. In a further embodiment, said second plant comprises another plant of variety ‘PAN2020’. In a different further embodiment, said second plant is a plant of a different variety than ‘PAN2020’. Another further embodiment relates to a Cannabis plant produced by cultivating the harvested F₁ seed or embryo produced by a method comprises crossing a Cannabis plant of variety ‘PAN2020’ with a second plant and harvesting the resultant F₁ seed or embryo.

A further embodiment relates to a method for producing an F₁ Cannabis seed, wherein the method comprises crossing a ‘PAN2020’ plant with a different Cannabis plant variety and harvesting the resultant F₁ Cannabis seed.

A further embodiment relates to a method for developing a Cannabis plant in a Cannabis plant breeding program, comprising applying plant breeding techniques. In further embodiments plant breeding techniques include recurrent selection, backcrossing, pedigree breeding, marker enhanced selection, or transformation to the Cannabis plant of ‘PAN2020’, or its parts, wherein application of said techniques results in development of a new Cannabis plant variety.

Another embodiment relates to a method of producing an F₁ seed or embryo, wherein the method comprises crossing a Cannabis plant of variety ‘HURV19PAN’ with a second plant and harvesting the resultant F₁ seed or embryo. In a further embodiment, said second plant comprises another plant of variety ‘HURV19PAN’. In a different further embodiment, said second plant is a plant of a different variety than ‘HURV19PAN’. Another further embodiment relates to a Cannabis plant produced by cultivating the harvested F₁ seed or embryo produced by a method comprises crossing a Cannabis plant of variety ‘HURV19PAN’ with a second plant and harvesting the resultant F₁ seed or embryo.

A further embodiment relates to a method for producing an F₁ Cannabis seed, wherein the method comprises crossing a ‘HURV19PAN’ plant with a different Cannabis plant variety and harvesting the resultant F₁ Cannabis seed.

A further embodiment relates to a method for developing a Cannabis plant in a Cannabis plant breeding program, comprising applying plant breeding techniques. In further embodiments plant breeding techniques include recurrent selection, backcrossing, pedigree breeding, marker enhanced selection, or transformation to the Cannabis plant of ‘HURV19PAN’, or its parts, wherein application of said techniques results in development of a new Cannabis plant variety.

Another embodiment relates to a method of producing an F₁ seed or embryo, wherein the method comprises crossing a Cannabis plant of variety ‘HURV2019CKH’ with a second plant and harvesting the resultant F₁ seed or embryo. In a further embodiment, said second plant comprises another plant of variety ‘HURV2019CKH’. In a different further embodiment, said second plant is a plant of a different variety than ‘HURV2019CKH’. Another further embodiment relates to a Cannabis plant produced by cultivating the harvested F₁ seed or embryo produced by a method comprises crossing a Cannabis plant of variety ‘HURV2019CKH’ with a second plant and harvesting the resultant F₁ seed or embryo.

A further embodiment relates to a method for producing an F₁ Cannabis seed, wherein the method comprises crossing a ‘HURV2019CKH’ plant with a different Cannabis plant variety and harvesting the resultant F₁ Cannabis seed.

A further embodiment relates to a method for developing a Cannabis plant in a Cannabis plant breeding program, comprising applying plant breeding techniques. In further embodiments plant breeding techniques include recurrent selection, backcrossing, pedigree breeding, marker enhanced selection, or transformation to the Cannabis plant of ‘HURV2019CKH’, or its parts, wherein application of said techniques results in development of a new Cannabis plant variety.

Another embodiment relates to a Cannabis plant with high cannabigerol (CBG) content and lack of tetrahydrocannabinol (THC) and lack of cannabidol (CBD).

In certain embodiments, a first Cannabis plant of a variety selected from the group consisting of ‘PAN2020’, ‘HURV19PAN’, and ‘HURV2019CKH’, is crossed with a second Cannabis plant of a different variety selected from the group consisting of ‘PAN2020’, ‘HURV19PAN’, and ‘HURV2019CKH’ to produce an F₁ seed or embryo and the resultant F₁ seed or embryo is harvested. In particular embodiments, a first Cannabis plant of variety ‘PAN2020’ is crossed with a second Cannabis plant of variety ‘HURV19PAN’. In other embodiments, a first Cannabis plant of variety ‘PAN2020’ is crossed with a second Cannabis plant of variety ‘HURV2019CKH’. In additional embodiments, a first Cannabis plant of variety ‘HURV19PAN’ is crossed with a second Cannabis plant of variety ‘HURV2019CKH’.

Another embodiment relates to Cannabis plants, plant parts, plant tissues and/or plant cells which comprise a CBG content that is about 10% or greater, a THC content of less than 0.3%, and a CBD content of less than 0.3%, based on the dry weight of plant inflorescences. In further embodiments, the Cannabis plants, plant parts, plant tissues and/or plant cells comprises CBG content of greater than about 10%, about 11%, about 12%, about 13%, about 14%, about 15%, about 16%, about 17%, about 18%, about 19%, or about 20%. In further embodiments, said Cannabis plants, plant parts, plant tissues and/or plant cells comprises a CBG content of about 10%, about 11%, about 12%, about 13%, about 14%, about 15%, about 16%, about 17%, about 18%, about 19%, or about 20%. In certain embodiments, the Cannabis plants, plant parts, plant tissues and/or plant cells comprises a THC content of less than about 0.3%, about 0.2%, about 0.1%, about 0.09%, about 0.08%, about 0.07%, about 0.06%, about 0.05%, about 0.04%, about 0.03%, about 0.02%, or about 0.01%. In further embodiments, said Cannabis plants, plant parts, plant tissues and/or plant cells comprises a THC content of about 0.3%, about 0.2%, about 0.1%, about 0.09%, about 0.08%, about 0.07%, about 0.06%, about 0.05%, about 0.04%, about 0.03%, about 0.02%, or about 0.01%. In further embodiments, said Cannabis plants, plant parts, plant tissues and/or plant cells comprises a THC content that is absent or undetectable. In further embodiments, said Cannabis plants, plant parts, plant tissues and/or plant cells comprises a CBD content of less than about 0.3%, about 0.2%, about 0.1%, about 0.09%, about 0.08%, about 0.07%, about 0.06%, about 0.05%, about 0.04%, about 0.03%, about 0.02%, or about 0.01%. In further embodiments, said Cannabis plants, plant parts, plant tissues and/or plant cells comprises a CBD content of about 0.3%, about 0.2%, about 0.1%, about 0.09%, about 0.08%, about 0.07%, about 0.06%, about 0.05%, about 0.04%, about 0.03%, about 0.02%, or about 0.01%. In further embodiments, said Cannabis plants, plant parts, plant tissues and/or plant cells comprises a CBD content that is absent or undetectable.

Another embodiment relates to a method for developing a Cannabis plant variety with a cannabinoid profile of high cannabigerol (CBG) content and lack of tetrahydrocannabinol (THC) and cannabidol (CBD). In certain embodiments, the method for developing said Cannabis plant comprises utilizing one or more of the following varieties in a breeding program: ‘Santhica 70’, ‘Antal’, ‘Zenit’, ‘KC Virtus’, ‘PAN2020’, ‘HURV19PAN’, or ‘HURV2019CKH’

Another embodiment relates to a method for developing a Cannabis plant variety which comprises a CBG content that is about 10% or greater, a THC content of less than 0.3%, and a CBD content of less than 0.3%, based on the dry weight of plant inflorescences. In further embodiments, the Cannabis plant variety comprises CBG content of greater than about 10%, about 11%, about 12%, about 13%, about 14%, about 15%, about 16%, about 17%, about 18%, about 19%, or about 20%. In further embodiments, said Cannabis plant variety comprises a CBG content of about 10%, about 11%, about 12%, about 13%, about 14%, about 15%, about 16%, about 17%, about 18%, about 19%, or about 20%. In certain embodiments, said Cannabis plant variety comprises a THC content of less than about 0.3%, about 0.2%, about 0.1%, about 0.09%, about 0.08%, about 0.07%, about 0.06%, about 0.05%, about 0.04%, about 0.03%, about 0.02%, or about 0.01%. In further embodiments, said Cannabis plant variety comprises a THC content of about 0.3%, about 0.2%, about 0.1%, about 0.09%, about 0.08%, about 0.07%, about 0.06%, about 0.05%, about 0.04%, about 0.03%, about 0.02%, or about 0.01%. In further embodiments, said Cannabis plant variety comprises a THC content that is absent or undetectable. In further embodiments, said Cannabis plant variety comprises a CBD content of less than about 0.3%, about 0.2%, about 0.1%, about 0.09%, about 0.08%, about 0.07%, about 0.06%, about 0.05%, about 0.04%, about 0.03%, about 0.02%, or about 0.01%. In further embodiments, said Cannabis plant variety comprises a CBD content of about 0.3%, about 0.2%, about 0.1%, about 0.09%, about 0.08%, about 0.07%, about 0.06%, about 0.05%, about 0.04%, about 0.03%, about 0.02%, or about 0.01%. In further embodiments, said Cannabis plant variety comprises a CBD content that is absent or undetectable.

In some embodiments, the cannabinoid contents of the Cannabis plants, plant parts, plant tissues or plant cells is measured using HPLC.

Other embodiments relate to extract from the Cannabis plants, plant parts, plant tissues or plant cells described herein. In some embodiments, the extract is selected from the group consisting of kief, hashish, bubble hash, solvent reduced oils, sludges, e-juice, and tinctures. In other embodiments, the extract retains the cannabinoid profile of the Cannabis plants, plant parts, plant tissues or plant cells from which it was made.

Other embodiments relate to edible product produced from the Cannabis plants, plant parts, plant tissues or plant cells described herein.

Other embodiments relate to compressed Cannabis pellet for smoking or vaporization, wherein the pellet comprises the Cannabis plants, plant parts, plant tissues or plant cells described herein. In some embodiments, the compressed Cannabis pellet comprises extracts from the Cannabis plants, plant parts, plant tissues or plant cells described herein. In some embodiments, the compressed Cannabis pellet is in the shape of a truncated cone or in the shape of a donut.

In addition to the exemplary aspects and embodiments described above, further aspects and embodiments will become apparent by study of the following descriptions.

### Definitions

“Allele” is any of one or more alternative forms for a gene.

As used herein, “gene” refers to a segment of nucleic acid.

A “locus” is the position or location of a gene on a chromosome.

As used herein “plant” refers to plants in the genus of Cannabis and plants derived thereof. Such as Cannabis plants produced via asexual reproduction and via seed production.

By “plant parts” or “Cannabis plant part” or “a part thereof” is meant to refer to any part of the plant and includes but is not limited to plant calli, plant clumps, plant protoplast, plant cells, embryos, protoplasts, meristematic cells, callus, pollen, stipule, leaf, petal, ovules, bract, trichome, branch, internode, bark, pubescence, tiller, rhizone, frond, blade, anthers, cotyledons, hypocotyl, pistils, roots, root tips, fruit, inflorescences, flowers, flower buds, seeds, shoot, petiole, or stems.

As used herein, “progeny” is the descendants of one or more of the parental lines and includes an F₁ Cannabis plant produced from the cross of two Cannabis plants where at least one plant includes a Cannabis plant disclosed herein and progeny further includes, but is not limited to, subsequent F₂, F₃, F₄, F₅, F₆, F₇, F₈, F₉, and F₁₀ generational crosses with the recurrent parental line.

As used herein, “regeneration” refers to the development of a plant from tissue culture or cell culture.

As used herein, “single gene converted plants” or “single gene conversion plants” or “backcross conversion plants” or “backcross converted plants” refers to plants which are developed by a plant breeding technique called backcrossing wherein essentially all of the desired morphological and physiological characteristics of a variety are recovered via the backcrossing technique in addition to the single gene transferred into the variety via the initial cross or via genetic engineering.

As used herein, “desired trait(s)” or “desired characteristic(s)” or “desired attribute(s)” or “desired gene(s)” or “desired phenotype(s)” refers to a phenotypical characteristic or genomic characteristic which is identified in a plant. For example, such a phenotypical characteristic or genomic characteristic may be a cannabinoid profile.

As used herein, “cannabinoid profile” refers to amount of one or more cannabinoids in a given plant, such as cannabidiol (CBD), cannabichromene, cannabigerol (CBG), Δ-9-tetrahydrocannabinol (THC), and cannabinol. For example, a cannabinoid profile of a plant is cannabigerol (CBG) as the predominant cannabinoid without the presence of tetrahydrocannabinol (THC) and without the presence of cannabidiol (CBD). Further, as used herein, content percentage of an identified cannabinoid, such as CBG, CBD, and THC, is the content as a % of dry weight of plant inflorescences.

As used herein, “sport” or “spontaneous mutation” or “natural mutation” refers to a mutation which has arisen spontaneously and has not been induced. These mutations may be selected from the initial variety and cultivated to produce an essentially derived variety. Sports, spontaneous mutations, and natural mutations may occur in an individual plant or on a plant part of the initial variety plant.

As used herein, “essentially derived variety” refers to the definitions set forth under 7 U.S.C. § 2401 and UPOV Convention.

As used herein, “crossing” may refer to comprising a simple x by y cross or the process of backcrossing depending on the context and may include additional tools or methods, such as genetic markers.

As used herein, the term “about” refers to a number that differs from the given number by less than 10%. In other embodiments, the term “about” indicates that the number differs from the given number by less than 9%, 8%, 7%, 6%, 5%, 4%, 3%, 2%, or 1%.

### BRIEF DESCRIPTION OF THE DRAWINGS

The accompanying photographs of FIG. 1 to FIG. 12 illustrate features of the ‘PAN2020’ variety. Additionally, experimental data obtained from extracts of the new varietal illustrate unique features of the plant, specifically its cannabinoid profile. Colors in the photographs may differ slightly from the color values cited in the botanical description.

FIGS. 1 and 2 depict the appearance of the whole plant during growth.

FIG. 3 depicts a top view of a lateral branch.

FIG. 4 depicts a bottom view of a lateral branch.

FIG. 5 depicts flowers of the plant.

FIGS. 6 and 7 depict distinct fan-shaped leaves, shown with a ruler to indicate scale.

FIG. 8 depicts small leaves of the plant, shown with a ruler to indicate scale.

FIG. 9 depicts a close-up detail of a flower, shown with a ruler to indicate scale.

FIG. 10 depicts a close-up detail of a flower, shown with a ruler to indicate scale.

FIG. 11 depicts two flower leaves, shown with a ruler to indicate scale.

FIG. 12 depicts a lateral branch of the plant.

FIG. 13 shows a chromatogram of a first sample extracted from the ‘PAN2020’ plant.

FIG. 14 shows a chromatogram of a second sample extracted from the ‘PAN2020’ plant.

FIG. 15 shows the breeding scheme of the ‘PAN2020’.

FIG. 16 shows a diagram of breeding of ‘HURV19PAN’.

FIG. 17 shows a diagram of breeding of ‘HURV2019CKH’.

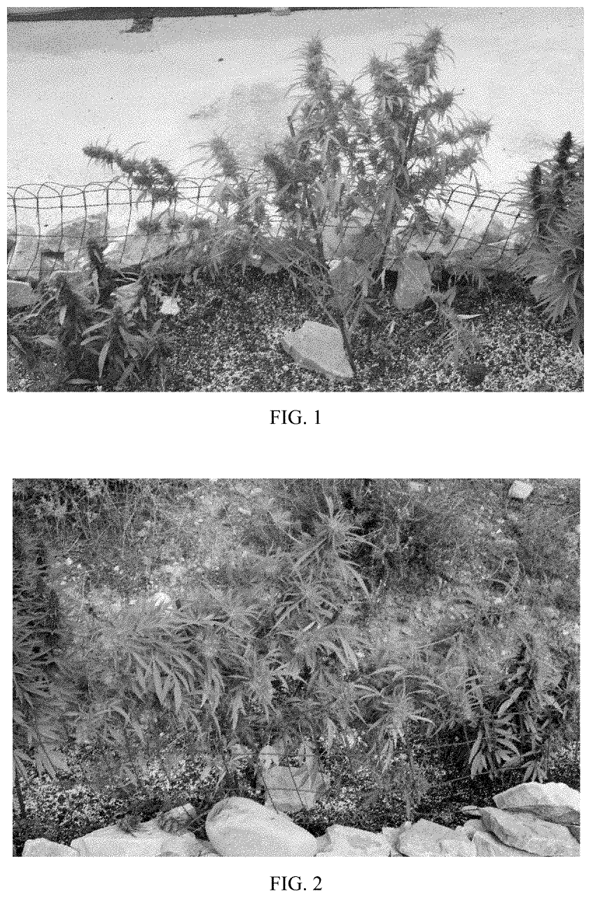

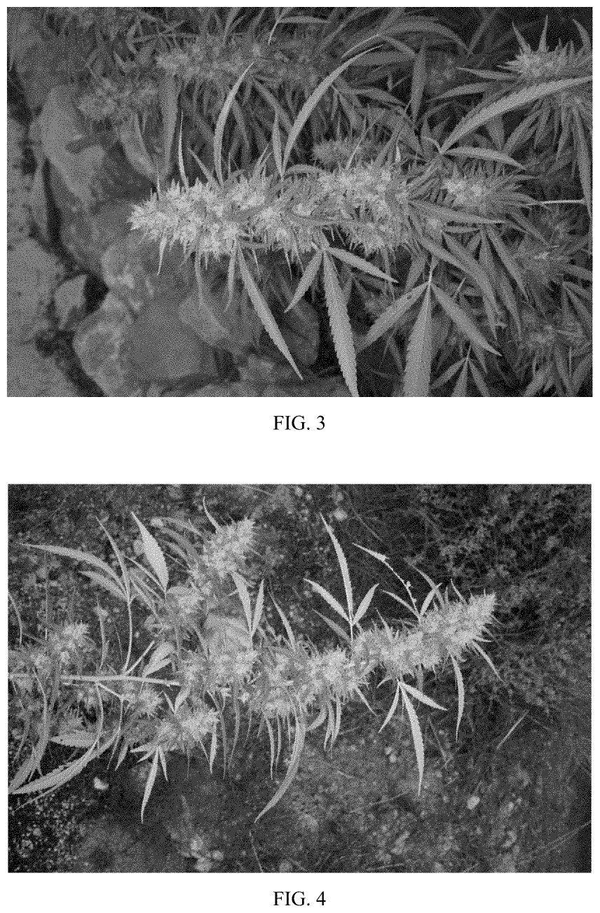

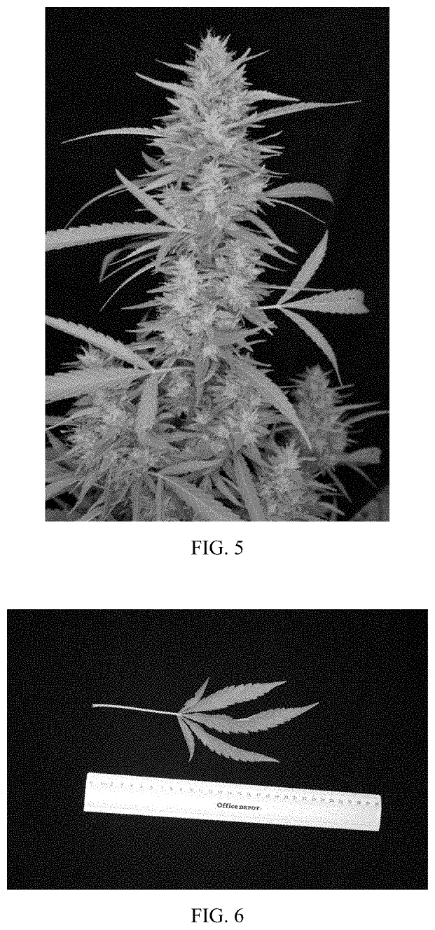

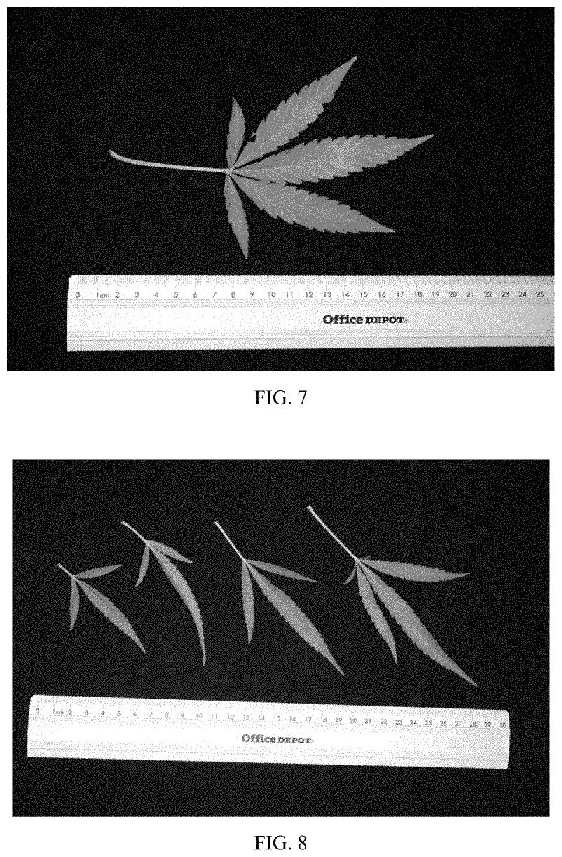

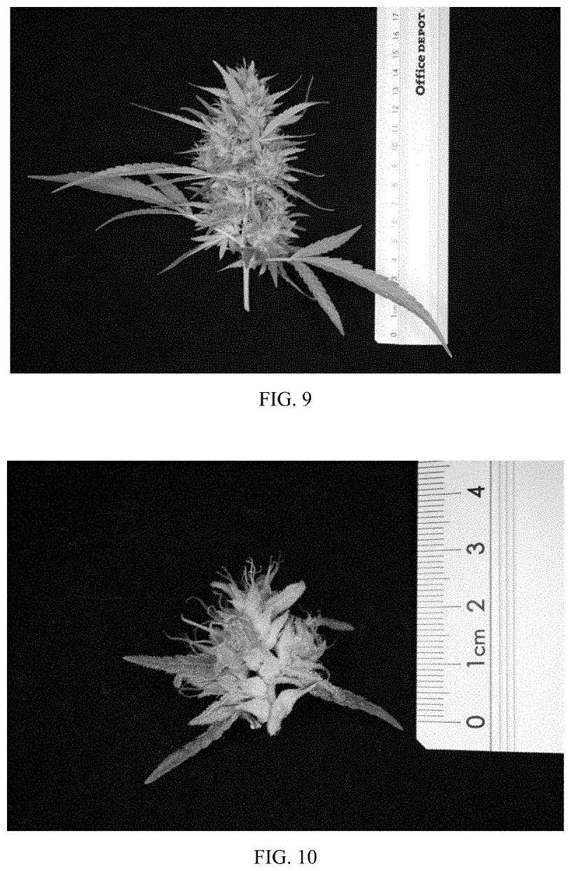

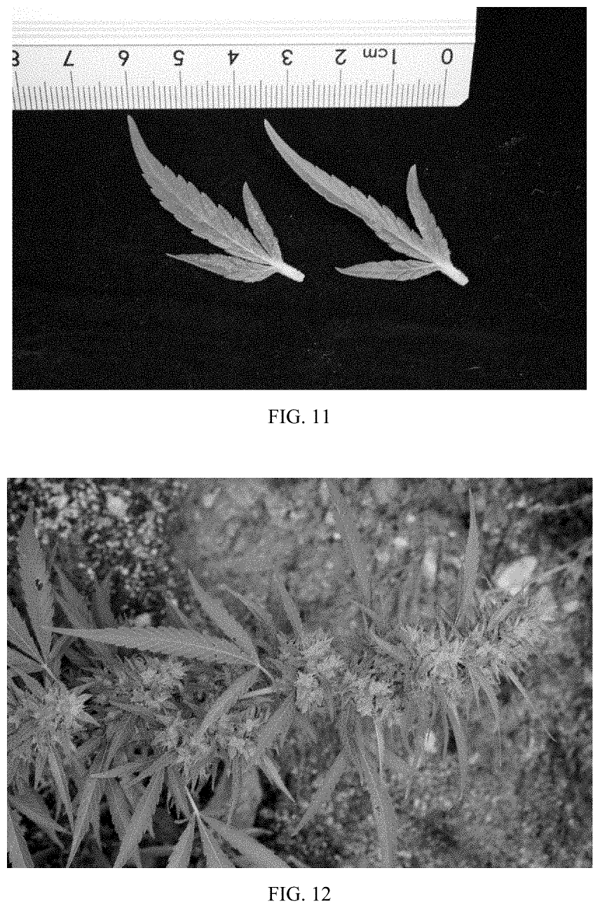

### DETAILED DESCRIPTION

### Origin of ‘PAN2020’

Variety PAN2020 was developed in Valencia, Spain. This new variety was developed under the project “Obtención de variedades de cáñamo (Cannabis sativa var. sativa) con elevado contenido en fitocannabinoides de interes terapeutico”

Briefly, a screening was performed to evaluate somaclonal variation ability of a collection of Cannabis sativa. An individual plant was selected by its high load capacity for callus induction and plant regeneration from leaves and cotyledons segments. An unusual cannabinoid profile (cannabigerol as predominant cannabinoid without the presence of tetrahydrocannabinol) was identified in one of the regenerated somaclones. In order to increase cannabigerol accumulation, the somaclone was crossed with a resinous individual and the resulting F2 progeny was evaluated. The individual plant with the greatest cannabinoid accumulation was then selected.

Latin name of genus and species

Cannabis sativa (L.)

Variety denomination

PAN2020

Parentage

The new variety is the result of multiple generational crosses originally using ‘KC VIRTUS’ (not patented) and ‘ZENIT’ (not patented). To develop the new PAN2020 variety, a screening was first performed to evaluate somaclonal variation ability in ‘KC VIRTUS’. An individual plant from the ‘KC VIRTUS’ somaclonal variation screen was selected by its high load capacity for callus induction and plant regeneration from leaves and cotyledons segments. This individual plant underwent callus induction and plant regeneration and then cannabinoid profile screening was conducted on the regenerated plants. An unusual cannabinoid profile (cannabigerol (CBG) as predominant cannabinoid without the presence of tetrahydrocannabinol (THC)) was identified in one of the regenerated somaclones. This selected regenerated somaclone was then crossed with a masculinized female plant selected from an F2 cross from ‘ZENIT’ x ‘ZENIT’. The resulting F1 progeny were crossed to create an F2 progeny and individual plants within that F2 progeny were evaluated. The individual plant with the greatest cannabinoid accumulation and the desired cannabinoid profile was then selected as the new variety. FIG. 15 outlines the detailed breeding scheme.

### Detailed Botanical Description of ‘PAN2020’

Following is a detailed description of the botanical and analytical chemical characteristics of ‘PAN2020’. The information for this botanical description was either collected or verified during the growing seasons of 2018-2019 in the growing areas of Valencia, Spain.

It should be noted that botanical characteristics, and to a lesser degree the analytical characteristics, are somewhat dependent on cultural practices and climatic conditions and can vary with location or year.

Locality where Grown and Observed

Variety PAN2020 was developed in Valencia, Spain.

Plant Characteristics
Species. —Cannabis sativa (L.)
Plant life forms. —Annual, herbaceous, dioecious flowering shrub, with prolific lateral branching
Plant growth habitat—An upright, tap-rooted annual plant, forming fibrous roots when asexually propagated
Plant origin—Cross of two proprietary clones
Plant propagation—Asexually propagated by vegetative cuttings and cloning
Propagation ease—Easy
Time to initiate roots—10 days at 25° C. and 18 hours of light per day
Height—0.9-2.0 m
Width—0.6-1.4 m
Plant vigor—Medium
Time to harvest—12 weeks
Resistance to pests or diseases—Not tested
Genetic modification—No

Leaf Characteristics
Leaf arrangement—Alternate
Leaf shape—Palmately compound (digitate)
Leaf structure—Linear-lanceolate leaflets with glandular hairs
Leaf margins—Serrated
Leaf hairs—Present, sessile glandular trichomes
Leaf length with petiole at maturity—18-23 cm
Petiole length at maturity—4-9 cm
Petiole color—Pantone No. 7492 U
Petiole anthocyanin intensity—Weak
Petiole trichome type—Non-glandular, cystolithic and non-cystolithic
Stipule length at maturity—4 mm
Stipule shape—Acuminate
Stipule color—Pantone No. 583 U
Number of leaflets—3 to 5
Middle largest (longest) leaflet length—11-15 cm
Middle largest (longest) leaflet width—2-2.6 cm
Middle largest (longest) leaflet length/width ratio—15:2.6
Number of teeth of middle leaflet (average)—25
Leaf color (upper side)—Pantone No. 377 U
Leaf color (lower side)—Pantone No. 383 U
Leaf glossiness—Light
Vein/midrib shape—A central vein in each leaflet; oblique veins from the central vein to the tips of each serration of the margin
Vein/midrib color—Pantone No. 7492 U
Aroma—Low, spicy and woody; the major terpene is beta-Caryophyllene.

Stem Characteristics
Stem shape—Round
Stem diameter at base—1.5-3.0 cm
Stem color—Pantone No. 583U
Branch strength—Medium to weak, flexible
Stem's internode length—Medium
Stem's depth of grooves—Shallow
Stem's trichome type—Non-glandular, cystolithic and non-cystolithic
Stem's amount of pitch in cross-section—Thick

Inflorescence Characteristics
Flowering (blooming) habit—Elliptical shaped racemose inflorescence, made up of a cluster of false spikes with single flowers
Proportion of female plants—100%
Inflorescence position—Axillary and terminal
Flower arrangement—Overlapping, touching, congested
Number of flowers per plant—Thousands
Number of flowers per inflorescence—Approximately 1000
Flower shape—A small green bract enclosing the ovary, with two slender stigmas sticking out of the bract, without petals or sepals
Flower (individual pistilate) length—6 mm
Flower (compound cyme) diameter—60 mm
Flower fragrance—Not very strong, floral, spicy, and woody; the major terpene is beta-Caryophyllene.
Corolla—Absent
Bract shape—Urceolate
Bract color—Pantone No. 7496U
Bract size—9 mm on average
Bract trichome type—Glandular capitate-sessile and capitate-stalked, non-glandular cystolithic
Bracteole average size—6 mm on average
Bracteole shape—Beaked, urceolate
Bracteole trichome type—Glandular capitate-sessile and capitate-stalked, non-glandular cystolithic
Bracteole color—Pantone No. 584 U
Calyx—No defined calyx
Stigma shape—Slender, acuminate
Stigma length—8 mm
Stigma color—Pantone No. 5865 U
Trichome mature color—Pantone No. Cool grey 1 U
Trichome immature color—Cristal transparent
Terminal bud shape—Elliptical
Terminal bud color—Pantone No. 7496 U
Pedicel—Absent
Staminate shape—N/A
Pollen—Absent
Seed—4 mm; marbled achene, Pantone No. 469 U in color
Petals—Apetalus
Max THC content —Not detected
Max CBD content—Not detected
Max CB G content—14-17%
Other Characteristics
Time period offlowering/blooming—7-9 weeks
Hardiness of plant—Not tested
Breaking action—Flexible, elastic
Rooting rate after cutting/cloning—99
Flower shipping quality—High
Flower storage life—Long
Flower market use—Extracts, concentrates, tinctures, oils, topicals
Productivity of the flower (weight/plant)—250 g/plant

Analytical Data of ‘PAN2020’

Extracts from the presently disclosed ‘PAN2020’ variety were obtained and analyzed using gas chromatography techniques. The samples were tested using an Agilent 7820 gas chromatograph with a flame ionization detector (FID). The samples were each prepared using Prazepam as an internal standard.

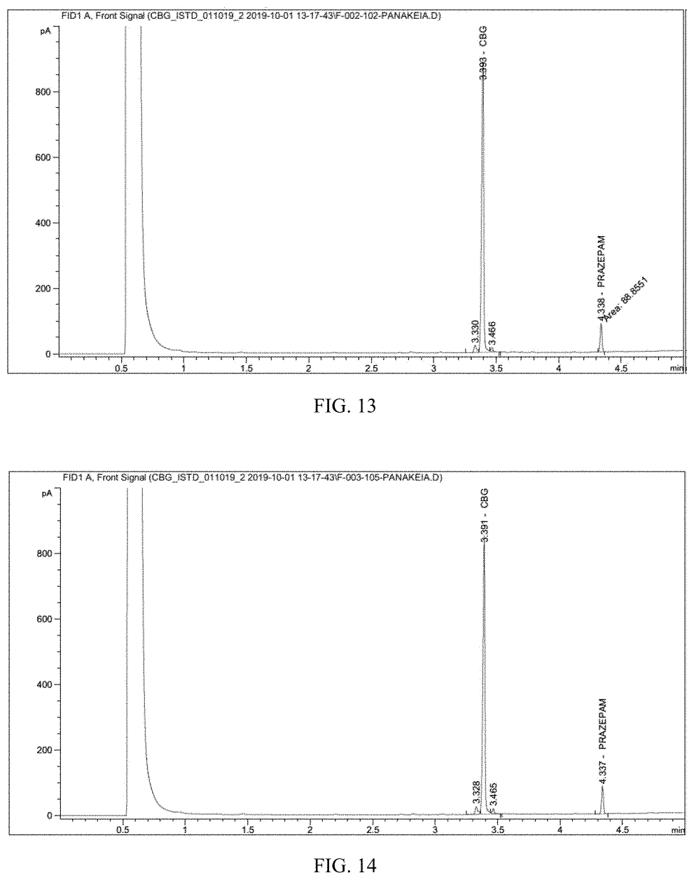

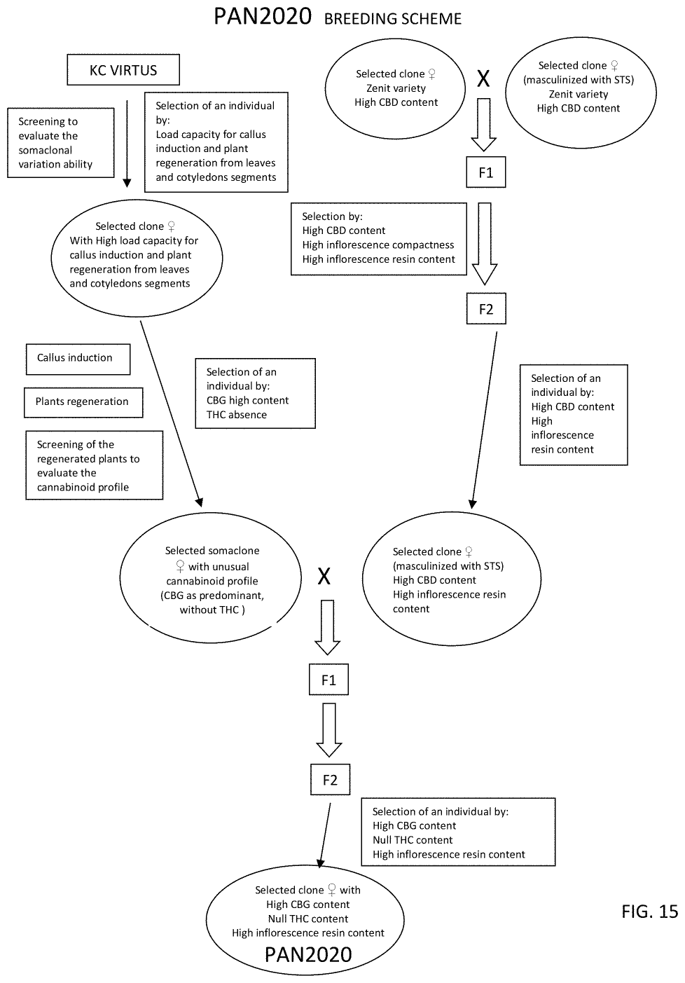

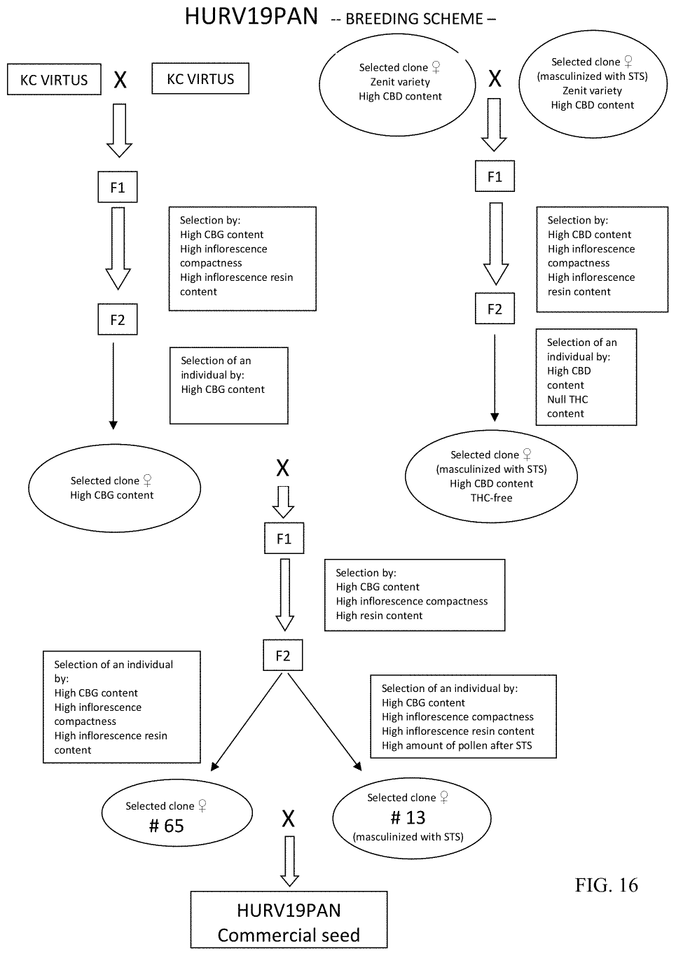

FIG. 13 shows a chromatogram of the data obtained from a first extract of the varietal and FIG. 14 shows of chromatogram of the data obtained from a second extract of the varietal. Table 1, shown below, illustrates the calculated area under the peaks shown in the chromatogram of FIG. 13 and Table 2 illustrates the calculated area under the peaks shown in FIG. 14.

TABLE 1
Area under the Peaks of Sample Chromatogram Shown in FIG. 13

| RetTime | | ISTD | Area | Amt/Area | Amount | | |
|---|---|---|---|---|---|---|---|
| [min] | Type | used | [pA*s] | ratio | % | Grp | Name |
| 2.896 | | 1 | — | — | — | | CBD |
| 3.304 | | 1 | — | — | — | | THC |
| 3.393 | VV R+ | 1 | 1059.28174 | 1.00250 | 17.911460 | | CBG |
| 3.571 | | 1 | — | — | — | | CBN |
| 4.338 | MF I | 1 | 88.85506 | 1.00000 | 0.014987 | | PRAZEPAM |

| | |
|---|---|
| Totals without ISTD(s): | 17.911460 |

As shown in FIG. 13 and Table 1, the first sample tested contained approximately 17.91% CBG.

TABLE 2
Area under the Peaks of Sample Chromatogram Shown in FIG. 14

| RetTime | | ISTD | Area | Amt/Area | Amount | | |
|---|---|---|---|---|---|---|---|
| [min] | Type | used | [pA*s] | ratio | % | Grp | Name |
| 2.896 | | 1 | — | — | — | | CBD |
| 3.304 | | 1 | — | — | — | | THC |
| 3.391 | VV R+ | 1 | 973.45337 | 1.00305 | 17.054502 | | CBG |
| 3.571 | | 1 | — | — | — | | CBN |
| 4.337 | BB I | 1 | 86.11620 | 1.00000 | 0.015041 | | PRAZEPAM |

| | |
|---|---|
| Totals without ISTD(s): | 17.054502 |

As shown in FIG. 14 and Table 2, the second sample tested contained approximately 17.05% CBG.

The presently disclosed variety thus has a much higher CBG content than previously known varieties of Cannabis sativa (L.), making it a promising candidate as a new source of CBG. Additionally, the unique cannabinoid profile of this variety could prove useful in medical applications as well as for other possible applications.

Propagation Status of ‘PAN2020’

Asexual plant propagation has been demonstrated for the disclosed varietal at Valencia, Spain. Specifically, from a selected individual plant, stem portions were cut with at least two knots with axillary shoots. The bark of the lower portion was slightly scraped to expose the cambium. The lower portion of the cuttings was introduced into a solution containing the auxins NAA and IBA (naphthaleacetic acid and indolbutyric acid). The cuttings were introduced into a rooting substrate (peat) previously moistened with water of low electrical conductivity (<0.1 mS/cm) to field capacity, leaving buried at least one of the nodes. The cuttings were placed under conditions of low illuminance (500 lux) and high relative humidity (>90%) with a photoperiod of 18/6 hours (i.e., 18 hours of light followed by six hours of darkness). To promote the emission of roots, a background heat source was placed so that the buried part of the cuttings was kept at a temperature 3 degrees higher than that of the aerial part. The material was sprayed regularly with water of low electrical conductivity (<0.1 mS/cm) and in 10 days at 25° C., the first roots were seen.

An assay growing 20 clones of the new variety was performed in indoor conditions (18 hours light/6 hour dark, at 60% relative humidity and 25° C.). Two months after, the photoperiod was changed to 12 hours light followed by 12 hours dark to induce plants flowering. All plants needed 60 days to finish the flowering stage. Analysis of cannabinoid content indicated that all plants accumulated cannabigerol (CBG) as the predominant cannabinoid without the presence of tetrahydrocannabinol (THC). Thus, the clones had the same properties as the parent.

Genetic stability of clones was analysed using 13 genomic Single Sequence Repeats (gSSR) markers (CSG01, CSG03, CSG05, CSG10, CSG12, CSG13, CSG14, CSG15, CSG18, CSG20, CSG22, CSG24 and CSG25) and the methodology described in Soler, S. et al., Use of embryos extracted from individual Cannabis sativa seeds for genetic studies and forensic applications. J. Forensic Sci. 61, 494-500 (2016) and Soler, S. et al., Genetic structure of Cannabis sativa var. indica cultivars based on genomic SSR (gSSR) markers: Implications for breeding and germplasm management, Industrial Crops & Products, 104, 171-178 (2017). The experimental results indicated a total genetic stability in all clones tested.

### Origin of ‘HURV19PAN’

A breeding scheme of ‘HURV19PAN’ is shown at FIG. 16. Briefly, ‘HURV19PAN’ is the result of multiple generational crosses originally using the public hemp varieties ‘KC Virtus’ and ‘Zenit’. More specifically, the great-grandparents of ‘HURV19PAN’ were a female plant selected from an F2 cross from ‘KC VIRTUS’ x ‘KC VIRTUS’ and a masculinized female plant selected from an F2 cross from ‘ZENIT’ x ‘ZENIT’.

Following the cross of these great-grandparent plants, two F1 plants were selected (grandparents) and crossed to create an F2 population. From that F2 population, two parental plants were selected, namely Selected clone No. 65 and Selected clone No. 13. These parental plants have been maintained through asexual propagation to allow ‘HURV19PAN’ seed to be repeatedly produced.

To produce ‘HURV19PAN’ seed, Selected clone No. 65 is used as the female parent and Selected clone No. 13 is used as the male parent (which was generated by taking the female plant and applying a silver thiosulfate treatment to produce a masculinized female plant [phenotypically male, but genotypically female]). The ‘HURV19PAN’ variety would not generally be considered a first-generation hybrid since the parents come from the same F2 cross as opposed to two distinct lines.

The criteria of high CBG (cannabigerol) content, as well as high compactness of the inflorescences and high resin content of female inflorescences was used for selection of progeny from the ‘KC Virtus’ x ‘KC Virtus’ cross.

The criteria of high CBD (cannabidiol) content, as well as high compactness of the inflorescences and high resin content of female inflorescences was used for selection of progeny from the ‘Zenif’ x ‘Zenif’ cross.

The criteria of high CBG and low THC content, as well as high compactness of the inflorescences and high resin content of female inflorescences was used for selection of the progeny derived from the cross between the selected individuals from the two previous lines (‘KC Virtus’ x ‘KC Virtus’ and ‘Zenif’ x ‘Zenit’).

The ‘HURV19PAN’ variety is produced by the direct crossing of two asexually propagated plants (i.e., Selected clone #65 (female) and Selected clone #13 (masculinized female)).

The parents of ‘HURV19PAN’ have maintained their botanical qualities and characteristics in 3 vegetative reproduction cycles, wherein each asexually vegetative reproduction cycle included 100 individuals. The asexually reproduced plants of Selected clone #65 and Selected clone #13 were tested by chromatography and PCR and it was observed that all the individuals of each vegetative reproduction cycle had a CBG-predominant chemotype IV. Accordingly, the parents of HURV19PAN (i.e., Selected clone #65 and Selected clone #13) undergo asexual propagation in a true-to-type manner wherein the characteristics of each of the parent varieties are homogeneous, stable, and strictly transmissible by asexual propagation from one generation to another.

In addition, the cross of Selected clone #65 x Selected clone #13 was conducted three times and 100 progeny plants of ‘HURV19PAN’ were observed and tested in each of the crosses. In these three sexual propagation cycles, the progeny ‘HURV19PAN’ plants maintained their botanical qualities and characteristics, showing that the variety is stable and uniform.

### Detailed Botanical Description of ‘HURV19PAN’

Following is a detailed description of the botanical and analytical chemical characteristics of ‘HURV19PAN’. It should be noted that botanical characteristics, and to a lesser degree the analytical characteristics, are somewhat dependent on cultural practices and climatic conditions and can vary with location or year.

Traits
Sex—gynoecious
Branching level: very high
Inflorescence compactness: very high
Amount of resin: very high

Plant:
THC Content Based on Dry Weight Analysis—0%
Plant Type—Sexually Propagated
Proportion of Hermaphrodite (Bisexual) Plants—Low (<5%)
Proportion of Female Plants—High (>95%)
Proportion of Male Plants—Low (<5%)
Natural Plant Height (At Flowering)—Medium
Branching—Strong

Seedling
Cotyledon Shape—Broad Obovate
Cotyledon Color—Medium Green
Hypocotyl Intensity of Anthocyanin Coloration—Weak
Plant Anthocyanin Coloration of Crown (before flowering)—Absent or Very Weak

Stem
Main Stem Color—Medium Green
Main Stem Length of Internode—Medium
Main Stem Length of Internode Mean of 20 (cm)—10.2
Main Stem Thickness—Thick
Main Stem Depth of Grooves—Medium
Main Stem Pith in Cross-Section—Thick

Leaves
Leaf Intensity of Green Color—Medium
Leaf Length of Petiole—Medium
Leaf Length of Petiole Mean of 20 (cm)—9.7
Leaf Anthocyanin Color of Petiole—Weak
Leaf Number of Leaflets—Few (<7)
Central Leaflet Length—Medium
Central Leaflet Length Mean of 20 (cm)—26
Central Leaflet Width—Medium
Central Leaflet Width Mean of 20 (mm)—34

Inflorescence
Time of Male Flowering (50% of plants)—Medium
Inflorescence THC Content—Absent or Very Low
Flowering—flowering is under photoperiod control; it is a “short day” plant variety.

Seed
1000 Seed Weight (g)—17
Seed Color of Testa—light brown
Seed Marbling of Color—Medium
Seed Color of Testa Color Code (RHS/Munsell)—RHS colour code 164A, UPOV Group No. 59
Seed Shape—Ovate

Disease Resistance and Insect Resistance—not tested to date.

Uses—Pharmaceutical/Medicinal, Oil

Content of Phytocannabinoids
CBD: 0%
THC: 0%
CBG: 10-15%

### Origin of ‘HURV2019CKH’

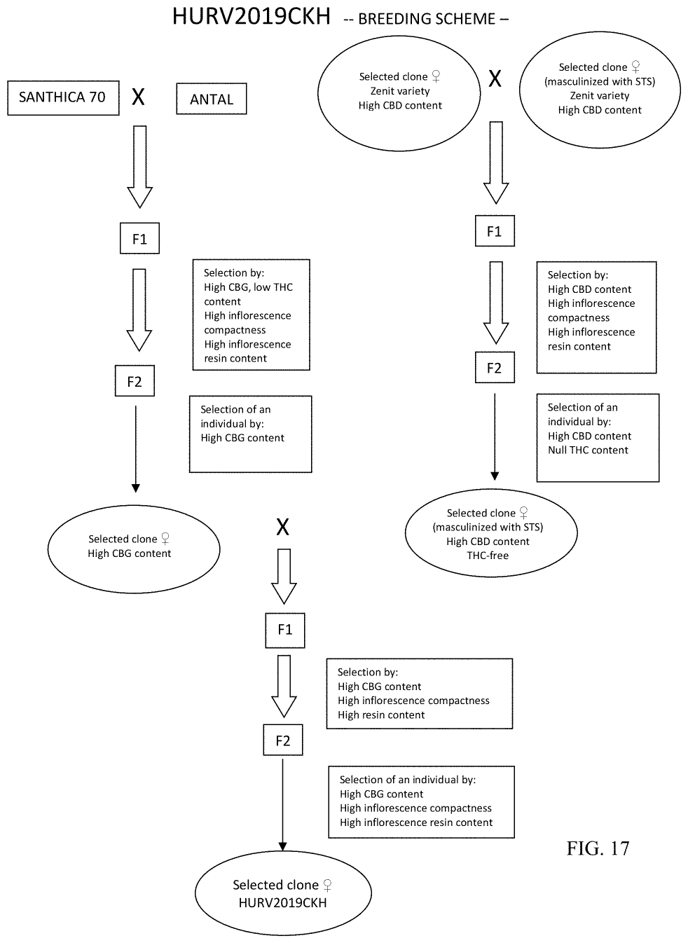

A breeding scheme of ‘HURV2019CKH’ is shown at FIG. 17. Briefly, the variety is the product of a cross of the varieties ‘Santhica 70’ and ‘Antal’. A selection from among the progeny of the selfed F1 generation was then crossed with a clone with a high content of CBD but lacking in THC. From among the selfed progeny of that cross, one clone with high CBG (cannabigerol) content was selected. The origin of the parental “clone with a high content of CBD” was selected from the selfed progeny of the cross of ‘Zenif’ x ‘Zenif’.

Additional selection criteria were compactness of the female inflorescence and resin content of the female inflorescence.

The ‘HURV2019CKH’ variety is uniform. An assay growing 20 clones of the new variety was performed in indoor conditions (18 hours light/6 hour dark, at 60% relative humidity and 25° C.). Two months after, the photoperiod was changed to 12 hours light followed by 12 hours dark to induce plants flowering. All plants needed 60 days to finish the flowering stage. Analysis of cannabinoid content indicated that all plants accumulated cannabigerol (CBG) as the predominant cannabinoid without the presence of tetrahydrocannabinol (THC). Thus, the clones had the same properties as the parent.

The ‘HURV2019CKH’ variety is stable. Genetic stability of clones was analyzed using 13 genomic Single Sequence Repeats (gSSR) markers (CSG01, CSG03, CSG05, CSG10, CSG12, CSG13, CSG14, CSG15, CSG18, CSG20, CSG22, CSG24 and CSG25) and the methodology described in Soler, S. et al., Use of embryos extracted from individual Cannabis sativa seeds for genetic studies and forensic applications. J. FORENSIC SCI. 61, 494-500 (2016) and Soler, S. et al., Genetic structure of Cannabis sativa var. indica cultivars based on genomic SSR (gSSR) markers: Implications for breeding and germplasm management, INDUSTRIAL CROPS & PRODUCTS, 104, 171-178 (2017). The experimental results indicated a total genetic stability in all clones tested.

### Detailed Botanical Description of ‘HURV2019CKH’

Following is a detailed description of the botanical and analytical chemical characteristics of ‘HURV2019CKH’. It should be noted that botanical characteristics, and to a lesser degree the analytical characteristics, are somewhat dependent on cultural practices and climatic conditions and can vary with location or year.

Traits
Sex—gynoecious
Branching level: very high
Inflorescence compactness: very high
Amount of resin: very high

Plant:
THC Content Based on Dry Weight Analysis—0%
Plant Type—Asexually Propagated
Proportion of Hermaphrodite (Bisexual) Plants—Low (<5%)
Proportion of Female Plants—High (>95%)
Proportion of Male Plants—Low (<5%)
Natural Plant Height (At Flowering)—Medium
Branching—Strong

Seedling
Cotyledon Shape—Broad Obovate
Cotyledon Color—Medium Green
Hypocotyl Intensity of Anthocyanin Coloration—Weak
Plant Anthocyanin Coloration of Crown (before flowering)—Absent or Very Weak

Stem
Main Stem Color—Medium Green
Main Stem Length of Internode—Medium
Main Stem Length of Internode Mean of 20 (cm)—7.5
Main Stem Thickness—Thick
Main Stem Depth of Grooves—Shallow
Main Stem Pith in Cross-Section—Thick

Leaves
Leaf Intensity of Green Color—Medium
Leaf Length of Petiole—Medium
Leaf Length of Petiole Mean of 20 (cm)—10.2
Leaf Anthocyanin Color of Petiole—Medium
Leaf Number of Leaflets—Few (<7)
Central Leaflet Length—Medium
Central Leaflet Length Mean of 20 (cm)—26.7
Central Leaflet Width—Medium
Central Leaflet Width Mean of 20 (mm)—36

Female Inflorescence
Inflorescence compactness—very high
Amount of Resin in the inflorescence—very high
Node density—dense Seed—none observed

Disease Resistance and Insect Resistance—not tested to date.

Uses—Pharmaceutical/Medicinal, Oil

Content of phytocannabinoids
CBD: 0%
THC: 0%
CBG: 13-17%

### Distinguishing Characteristics of ‘PAN2020’, ‘HURV19PAN’, and ‘HURV2019CKH’

The ‘PAN2020’ variety of the present invention can readily be distinguished from its ancestors. More specifically, ‘KC VIRTUS’ (i.e., the seed grandparent), ‘ZENIT’ (i.e., the pollen grandparent), the male parent (i.e., resinous individual), and the female parent (i.e., selected regenerated somaclone) provide a different chemotype and cannabinoid content compared to the ‘PAN2020’, as shown in the Table 3, below. These ancestors can also be readily distinguished from ‘HURV19PAN’ and ‘HURV2019CKH’ as shown in Table 3.

TABLE 3

| Variety | CBG Content | THC Content | CBD Content |
|---|---|---|---|
| ‘KC VIRTUS’ | LOW | LOW | LOW |
| ‘ZENIT’ | | | HIGH |
| Male Parent | LOW | LOW | HIGH |
| of ‘PAN2020’ | | | |
| (resinous individual) | | | |
| Female Parent | MEDIUM | NULL | LOW |
| of ‘PAN2020’ | | | |
| (selected | | | |
| regenerated | | | |
| somaclone) | | | |
| ‘PAN2020’ | HIGH (~17%) | NULL | NULL |
| ‘HURV19PAN’ | HIGH (~10-15%) | NULL | NULL |
| ‘HURV2019CKH’ | HIGH (~13-17%) | NULL | NULL |

Moreover, ‘PAN2020’, ‘HURV19PAN’, and ‘HURV2019CKH’ can readily be distinguished from related similar non-parental/grandparental varieties due to their high CBG content and total absence of THC. These new Cannabis sativa varieties ‘PAN2020’, ‘HURV19PAN’, and ‘HURV2019CKH’ can be distinguished from all other known Cannabis varieties known to the Inventor by its unusual cannabinoid profile. Specifically, ‘PAN2020’, ‘HURV19PAN’, and ‘HURV2019CKH’ each contain cannabigerol (CBG) as the predominant cannabinoid without the presence of tetrahydrocannabinol (THC) and without the presence of cannabidiol (CBD). For example, ‘HOLY CRUNCH’ (U.S. Pat. No. 31,874) has 6.6-16.7% THC, 6.5-15.3% CBD, and 0.25-1.9% CBG.

Breeding with Cannabis Varieties ‘PAN2020’, ‘HURV19PAN’, and/or ‘HURV2019CKH’

The goal of plant breeding is to develop new, unique, and superior plants. The breeder typically selects and crosses two or more parental lines, followed by selection, producing many new genetic combinations. The breeder can theoretically generate billions of different genetic combinations via crossing, selection, selfing, and/or mutagenesis.

Due to the large number of possible genetic combinations that result from such a cross, it is often difficult to reproduce a variety with a particular desired trait by simply crossing the same original parents and utilizing the same selection techniques. This unpredictability results in the expenditure of large amounts of research funds to develop superior Cannabis varieties. To advance breeding programs more quickly, breeders often use a variety that contains the desired trait as a parental line as a starting point rather than trying to recreate the variety possessing that desired trait.

Breeding programs combine desirable traits from two or more varieties or various broad-based sources into breeding pools from which varieties are developed and selected for desired phenotypes. Breeding programs may include artificial pollination wherein two parents are crossed which previously had been studied in the hope that the parents would contribute the desired characteristics. Seeds resulting from such an artificial pollination can be sown to obtain small plants, and then selective study can be used to identify a new plant variety which includes the desired phenotype.

Pedigree breeding is commonly used for the improvement of self-pollinating plants. An example of pedigree breeding is when two parents that possess favorable traits are crossed to produce an F₁. Then an F₂ population is produced by selfing one or several F₁s. This is followed by selection of the best individuals which may begin in the F₂ population; then, often beginning in the F₃, the best individuals in the best families are selected. Replicated testing of families may often begin in the F₄ generation to improve the effectiveness of selection for traits with low heritability. At an advanced stage of inbreeding, the best lines or mixtures of phenotypically similar lines may be further tested for selection of new varieties.

Using Cannabis Plant ‘PAN2020’, ‘HURV19PAN’, and/or ‘HURV2019CKH’ to Develop Other Plants

‘PAN2020’, ‘HURV19PAN’, and/or ‘HURV2019CKH’ plants can provide a source of breeding material that may be used to develop new Cannabis plants and varieties. In certain embodiments, two or more of the ‘PAN2020’, ‘HURV19PAN’, and ‘HURV2019CKH’ varieties can be used in a breeding program. For example, a breeding could utilize ‘PAN2020’ and ‘HURV19PAN’, ‘PAN2020’ and ‘HURV2019CKH’, or ‘HURV19PAN’ and ‘HURV2019CKH’ as sources of genetic material in a breeding program. For example, the two varieties could be used as parents in a cross, or could be utilized at two different generational levels within a breeding scheme. In other examples, all three of varieties ‘PAN2020’, ‘HURV19PAN’, and ‘HURV2019CKH’ could be used as sources of genetic material in a breeding program.

Plant breeding techniques known in the art and used in a Cannabis plant breeding program include, but are not limited to, recurrent selection, mass selection, bulk selection, hybridization, mass selection, backcrossing, pedigree breeding, open-pollination breeding, restriction fragment length polymorphism enhanced selection, genetic marker enhanced selection, mutagenesis, and transformation. Combinations of these techniques may be used. There are many analytical methods available to evaluate a new variety. The traditional method of analysis is the observation of phenotypic traits, but genotypic analysis may also be used.

Additional Breeding Methods

Any plants produced using a plant of variety ‘PAN2020’, ‘HURV19PAN’, or ‘HURV2019CKH’ as at least one parent are further embodiments of the present invention. Thus, plants which contain about 50% of the genetic composition of ‘PAN2020’, ‘HURV19PAN’, or ‘HURV2019CKH’ are another embodiment of the present invention. Methods for producing progeny using ‘PAN2020’, ‘HURV19PAN’, or ‘HURV2019CKH’ as at least one of the parents are well-known in the art and some of the more commonly used breeding methods are described herein. Descriptions of breeding methods are well-known in the art and may be found in several reference books.

Breeding steps that may be used in the ‘PAN2020’, ‘HURV19PAN’, and/or ‘HURV2019CKH’ plant breeding programs can include, for example, artificial pollination using ‘PAN2020’, ‘HURV19PAN’, or ‘HURV2019CKH’ as at least one of the parents, pedigree breeding, backcrossing, and recurrent selection. In conjunction with these steps, techniques such as mutagenesis, RFLP-enhanced selection, genetic marker enhanced selection (for example, SSR markers), and gene editing may be utilized.

Pedigree Breeding

Pedigree breeding starts with the crossing of two genotypes, such as ‘PAN2020’, ‘HURV19PAN’, or ‘HURV2019CKH’ and another different Cannabis variety having one or more desirable characteristics that is lacking or which complements the ‘PAN2020’, ‘HURV19PAN’, or ‘HURV2019CKH’ phenotype. If the two original parents do not provide all the desired characteristics, other sources can be included in the breeding population. In the pedigree method, superior plants are selfed and selected in successive filial generations. In the succeeding filial generations, the heterozygous condition gives way to homogeneous varieties as a result of self-pollination and selection. Typically in the pedigree method of breeding, five or more successive filial generations of selfing and selection is practiced: F₁ to F₂; F₂ to F₃; F₃ to F₄; F₄ to F₅; etc. After a sufficient amount of inbreeding, successive filial generations will serve to increase seed of the developed variety. Preferably, the developed variety comprises homozygous alleles at about 95% or more of its loci.

Backcross Breeding

Backcross breeding has been used to transfer genes for a simply inherited, highly heritable trait into a desirable homozygous variety or inbred line which is the recurrent parent. The source of the trait to be transferred is called the donor parent. After the initial cross, individuals possessing the phenotype of the donor parent are selected and repeatedly crossed (backcrossed) to the recurrent parent. The resulting plant is expected to have the attributes of the recurrent parent and the desirable trait transferred from the donor parent. This is also known as single gene conversion and/or backcross conversion.

In addition to being used to create a backcross conversion, backcrossing can also be used in combination with pedigree breeding. As discussed previously, backcrossing can be used to transfer one or more specifically desirable traits from one variety, the donor parent, to a developed variety called the recurrent parent, which has overall good commercial characteristics yet lacks that desirable trait or traits. However, the same procedure can be used to move the progeny toward the genotype of the recurrent parent, but at the same time retain many components of the nonrecurrent parent by stopping the backcrossing at an early stage and proceeding with selfing and selection. For example, a Cannabis plant, such as ‘PAN2020’, ‘HURV19PAN’, or ‘HURV2019CKH’ may be crossed with another variety to produce a first-generation progeny plant. The first-generation progeny plant may then be backcrossed to one of its parent varieties to create a BC₁ or BC₂. Progeny are selfed and selected so that the newly developed variety has many of the attributes of the recurrent parent and yet several of the desired attributes of the nonrecurrent parent. This approach leverages the value and strengths of the recurrent parent for use in new Cannabis varieties.

Therefore, another embodiment is a method of making a backcross conversion of ‘PAN2020’, comprising the steps of crossing ‘PAN2020’ with a donor plant comprising a desired trait, selecting an F₁ progeny plant comprising the desired trait, and backcrossing the selected F₁ progeny plant to ‘PAN2020’. This method may further comprise the step of obtaining a molecular marker profile of ‘PAN2020’ and using the molecular marker profile to select for a progeny plant with the desired trait and the molecular marker profile of ‘PAN2020’.

Therefore, another embodiment is a method of making a backcross conversion of ‘HURV19PAN’, comprising the steps of crossing ‘HURV19PAN’ with a donor plant comprising a desired trait, selecting an F₁ progeny plant comprising the desired trait, and backcrossing the selected F₁ progeny plant to ‘HURV19PAN’. This method may further comprise the step of obtaining a molecular marker profile of ‘HURV19PAN’ and using the molecular marker profile to select for a progeny plant with the desired trait and the molecular marker profile of ‘HURV19PAN’.

Therefore, another embodiment is a method of making a backcross conversion of ‘HURV2019CKH’, comprising the steps of crossing ‘HURV2019CKH’ with a donor plant comprising a desired trait, selecting an F₁ progeny plant comprising the desired trait, and backcrossing the selected F₁ progeny plant to ‘HURV2019CKH’. This method may further comprise the step of obtaining a molecular marker profile of ‘HURV2019CKH’ and using the molecular marker profile to select for a progeny plant with the desired trait and the molecular marker profile of ‘HURV2019CKH’.

Recurrent Selection and Mass Selection

Recurrent selection is a method used in a plant breeding program to improve a population of plants. ‘PAN2020’, ‘HURV19PAN’, and ‘HURV2019CKH’ are each suitable for use in a recurrent selection program. The method entails individual plants cross-pollinating with each other to form progeny. The progeny are grown and the superior progeny, which include individual plant, half-sib progeny, full-sib progeny, and selfed progeny, are selected by any number of selection methods. The selected progeny are cross-pollinated with each other to form progeny for another population. This population is planted and again superior plants are selected to cross-pollinate with each other. Recurrent selection is a cyclical process and therefore can be repeated as many times as desired. The objective of recurrent selection is to improve the traits of a population. The improved population can then be used as a source of breeding material to obtain new varieties for commercial or breeding use, including the production of a synthetic variety. A synthetic variety is the resultant progeny formed by the intercrossing of several selected varieties.

Mass selection is a useful technique especially when used in conjunction with molecular marker enhanced selection. In mass selection, seeds from individuals are selected based on phenotype or genotype. These selected seeds are then bulked and used to grow the next generation. Bulk selection requires growing a population of plants in a bulk plot, allowing the plants to self-pollinate, harvesting the seed in bulk, and then using a sample of the seed harvested in bulk to plant the next generation. Also, instead of self-pollination, directed pollination could be used as part of the breeding program.

Mass and recurrent selections can be used to improve populations of either self- or cross-pollinating plants. A genetically variable population of heterozygous individuals is either identified, or created, by intercrossing several different parents. The best plants are selected based on individual superiority, outstanding progeny, or excellent combining ability. The selected plants may then be intercrossed to produce a new population in which further cycles of selection are continued.

### Essentially Derived Varieties

Mutations that occur spontaneously or are artificially induced can be useful sources of variability for a plant breeder. An essentially derived variety is predominantly derived from the initial variety, or from a variety that is itself predominantly derived from the initial variety, while retaining the expression of the essential characteristics that result from the genotype or combination of genotypes of the initial variety; is clearly distinguishable from the initial variety; and except for the differences which result from the act of derivation, it conforms essentially to the initial variety in the expression of the essential characteristics that result from the genotype or combination of genotypes of the initial variety. An essentially derived variety may be obtained by the selection of a natural mutant (e.g., spontaneous mutant, also referred to as a sport) or induced mutant or of a somaclonal variant, the selection of a variant individual from plants of the initial variety, backcrossing, transformation by genetic engineering, or other methods.

Therefore, another embodiment is to an essentially derived variety of ‘PAN2020’. A further embodiment is to methods of artificially inducing an essentially derived variety from ‘PAN2020’.

Therefore, another embodiment is to an essentially derived variety of ‘HURV19PAN’. A further embodiment is to methods of artificially inducing an essentially derived variety from ‘HURV19PAN’.

Therefore, another embodiment is to an essentially derived variety of ‘HURV2019CKH’. A further embodiment is to methods of artificially inducing an essentially derived variety from ‘HURV2019CKH’.

Mutagenesis

The goal of artificial mutagenesis is to increase the rate of mutation for a desired characteristic. Mutation rates can be increased by many different means including, but not limited to temperature, long-term seed storage, tissue culture conditions, ionizing radiation, such as X-rays, Gamma rays (e.g., cobalt 60 or cesium 137), neutrons, (product of nuclear fission by uranium 235 in an atomic reactor), Beta radiation (emitted from radioisotopes such as phosphorus 32 or carbon 14), or ultraviolet radiation (preferably from 2500 to 2900 nm); chemical mutagens (such as base analogues (5-bromo-uracil)), related compounds (8-ethoxy caffeine), antibiotics (streptonigrin), alkylating agents (sulfur mustards, nitrogen mustards, epoxides, ethylenamines, sulfates, sulfonates such as ethyl methanesulfonate, sulfones, lactones), sodium azide, hydroxylamine, nitrous acid, methylnitrilsourea, or acridines; and/or TILLING (targeting induced local lesions in genomes), wherein mutation is induced typically by chemical mutagens and the mutagenesis is accompanied by the isolation of chromosomal DNA from the mutated plant lines or seeds and then screening of the population of the seeds or plants is performed at the DNA level using molecular techniques. Once a desired trait is observed through mutagenesis the trait may then further be incorporated into existing germplasm by traditional breeding techniques.

Details of breeding with mutagenesis or a mutant variety can be found, for example, in the following: Sikora, et al., Mutagenesis as a Tool in Plant Genetics, Functional Genomics, and Breeding, 2011 INTERNATIONAL JOURNAL OF PLANT GENOMICS, 13 pages; Petilino, Genome editing in plants via designed zinc finger nucleases (2015) IN VITRO CELL DEV BIOL PLANT. 51(1): 1-8; and Daboussi, et al., Engineering Meganuclease for Precise Plant Genome Modification, “Advances in New Technology for Targeted Modification of Plant Genomes” (2015). SPRINGER SCIENCE+BUSINESS. 21-38. In addition, mutations created in other Cannabis plants may be used to produce a backcross conversion using ‘PAN2020’, ‘HURV19PAN’, or ‘HURV2019CKH’

Gene Editing

Gene editing can be done through a variety of techniques including zinc finger nucleases and CRISPR/Cas9 technology. See e.g., Saunders & Joung, NATURE BIOTECHNOLOGY, 32, 347-355, 2014. CRISPR is a type of targeted genome editing system that stands for Clustered Regularly Interspaced Short Palindromic Repeats. This system and CRISPR-associated (Cas) genes naturally enable organisms, such as select bacteria and archaea, to respond to and eliminate invading genetic material. See e.g., Ishino, et al., J. BACTERIOL. 169, 5429-5433 (1987). CRISPR/Cas9 technology is used for direct gene editing, in vivo and in vitro. Many plants have already been modified using the CRISPR system. See e.g., International Publication No. WO2014/068346; Martinelli, et al., Proposal of a Genome Editing System for Genetic Resistance to Tomato Spotted Wilt Virus 2014 AMERICAN JOURNAL OF APPLIED SCIENCES; Noman, et al., CRISPR-Cas9: Tool for Qualitative and Quantitative Plant Genome Editing, November 2016 FRONTIERS IN PLANT SCIENCE Vol. 7; and Zhang et al., Exploiting the CRISPR/Cas9 System for Targeted Genome Mutagenesis in Petunia February 2016 SCIENCE REPORTS Volume 6.

Additional information about CRISPR/Cas9 system technology including crRNA-guided surveillance complex systems for gene editing may be found in the following documents: U.S. Application Publication No. 2010/0076057; U.S. Application Publication No. 2014/0179006; U.S. Pat. No. 10,000,772; U.S. Application Publication No. 2014/0294773; Sorek et al., ANNU. REV. BIOCHEM. 82:273-266, 2013; and Wang, S. et al., PLANT CELL REP (2015) 34: 1473-1476. Therefore, it is another embodiment to use gene editing, including the CRISPR/Cas9 system, on ‘PAN2020’, ‘HURV19PAN’, or ‘HURV2019CKH’ to modify traits, such as hardiness and resistances or tolerances to pests, herbicides, diseases, and viruses.

Introduction of a New Trait or Locus into ‘PAN2020’

‘PAN2020’, ‘HURV19PAN’, and ‘HURV2019CKH’ each represents a new base of genetics into which a new locus or trait may be introgressed or introduced. Direct transformation and backcrossing represent two important methods that can be used to accomplish such an introgression. The term backcross conversion and single locus conversion are used interchangeably to designate the product of a backcrossing program.

Transformation

Transformation methods include, but are not limited to, expression vectors introduced into plant tissues using a gene transfer method, such as microprojectile-mediated delivery, DNA injection, electroporation, and the like. In some embodiments, expression vectors are introduced into plant tissues of ‘PAN2020’ by using either microprojectile-mediated delivery with a biolistic device or by using Agrobacterium-mediated transformation. Accordingly, further embodiments are methods of transformation using ‘PAN2020’ and the transformant plants obtained with the protoplasm of the subject ‘PAN2020’ plants. In other embodiments, expression vectors are introduced into plant tissues of ‘HURV19PAN’ by using either microprojectile-mediated delivery with a biolistic device or by using Agrobacterium-mediated transformation. Accordingly, further embodiments are methods of transformation using ‘HURV19PAN’ and the transformant plants obtained with the protoplasm of the subject ‘HURV19PAN’ plants. In other embodiments, expression vectors are introduced into plant tissues of ‘HURV2019CKH’ by using either microprojectile-mediated delivery with a biolistic device or by using Agrobacterium-mediated transformation. Accordingly, further embodiments are methods of transformation using ‘HURV2019CKH’ and the transformant plants obtained with the protoplasm of the subject ‘HURV2019CKH’ plants.

Expression Vectors for ‘PAN2020’, ‘HURV19PAN’, and/or ‘HURV2019CKH’ Transformation: Marker Genes

Plant transformation typically involves the construction of an expression vector which will function in plant cells. Such expression vectors comprise DNA comprising a gene under control of, or operatively linked to, a regulatory element (e.g., a promoter). Expression vectors typically include at least one genetic marker operably linked to a regulatory element that allows transformed cells containing the marker to be either recovered by negative selection (e.g., inhibiting growth of cells that do not contain the selectable marker gene) or by positive selection (e.g., screening for the product encoded by the genetic marker). Commonly used selectable marker genes for plant transformation are well-known in the art, and include, for example, genes that code for enzymes that metabolically detoxify a selective chemical agent which may be an antibiotic or an herbicide, or genes that encode an altered target which is insensitive to the inhibitor. Positive selection methods are also known in the art.

One commonly used selectable marker gene for plant transformation is the neomycin phosphotransferase II (nptll) gene which, when under the control of plant regulatory signals, confers resistance to kanamycin. Another commonly used selectable marker gene is the hygromycin phosphotransferase gene which confers resistance to the antibiotic hygromycin.

Selectable marker genes for plant transformation not of bacterial origin include, for example, mouse dihydrofolate reductase, plant 5-enolpyruvylshikimate-3-phosphate synthase, and plant acetolactate synthase. See, e.g., Eichholtz, et al., SOMATIC CELL MOL. GENET., 13:67 (1987); Shah, et al., SCIENCE, 233:478 (1986); and Charest, et al., PLANT CELL REP., 8:643 (1990).

Another class of marker genes for plant transformation requires screening of presumptively transformed plant cells. Reporter genes are an example of this type of marker genes and can be used to quantify or visualize the spatial pattern of expression of a gene in specific tissues. Moreover, reporter genes can be fused to a gene or gene regulatory sequence for the investigation of gene expression. Commonly used marker genes for screening presumptively transformed cells include β-glucuronidase (GUS), β-galactosidase, luciferase, and chloramphenicol acetyltransferase. See, e.g., Teeri, et al., EMBO J., 8:343 (1989); Koncz, et al., PROC. NATL. ACAD. SCI. USA, 84:131 (1987); and DeBlock, et al., EMBO J., 3:1681 (1984).

Expression Vectors for ‘PAN2020’, ‘HURV19PAN’, and/or ‘HURV2019CKH’ Transformation: Promoters

Genes included in expression vectors must be driven by a nucleotide sequence comprising a regulatory element (e.g., a promoter). Many types of promoters are well known in the art, as are other regulatory elements that can be used alone or in combination with promoters.

Some promoters are under developmental control and include promoters that preferentially initiate transcription in certain tissues, such as leaves, roots, seeds, fibers, xylem vessels, tracheids, or sclerenchyma. Such promoters are typically referred to as “tissue-preferred.” Whereas promoters that initiate transcription only in a certain tissue are typically referred to as “tissue-specific.” A “cell-type” specific promoter primarily drives expression in certain cell types in one or more organs, for example, vascular cells in roots or leaves. Whereas an “inducible” promoter typically refers to a promoter which is under environmental control. Examples of environmental conditions that may affect transcription by inducible promoters include anaerobic conditions or the presence of light. Tissue-specific, tissue-preferred, cell-type specific, and inducible promoters constitute the class of “non-constitutive” promoters. A “constitutive” promoter refers to a promoter that is active under most environmental conditions. Many types of promoters are well known in the art.

Additional Transformation Embodiments

The foregoing methods for transformation may be used for producing a transgenic variety. The transgenic variety could then be crossed with another (non-transformed or transformed) variety in order to produce a new transgenic variety. Alternatively, a genetic trait that has been engineered into a particular Cannabis line using the foregoing transformation techniques could be moved into another line using traditional breeding techniques that are well known in the art. For example, a backcrossing approach could be used to move an engineered trait from a publicly available variety into an elite variety, such as ‘PAN2020’, ‘HURV19PAN’, or ‘HURV2019CKH’, or a backcrossing approach can be used to move a foreign gene from a variety containing the foreign gene in its genome into a variety that does not contain that gene.

Likewise, by means of such embodiments, commercially important genes can be expressed in transformed plants. More particularly, plants can be genetically engineered to express various phenotypes of commercial interest, including, but not limited to, genes that confer resistance to pests or disease, genes that confer resistance to an herbicide, genes that confer or contribute to a value-added or desired trait, genes that control male sterility, genes that create a site for site specific DNA integration, and genes that affect abiotic stress resistance. Many different genes are known and could potentially be introduced into a Cannabis plant according to the invention. Non-limiting examples of particular genes and corresponding phenotypes one may choose to introduce into a Cannabis plant include one or more genes for insect tolerance, such as a Bacillus thuringiensis (Bt.) gene, pest tolerance such as genes for fungal disease control, herbicide tolerance such as genes conferring glyphosate tolerance, and genes for quality improvements such as environmental or stress tolerances, or any desirable changes in plant physiology, growth, development, morphology, or plant product(s). For example, structural genes would include any gene that confers insect tolerance including but not limited to a Bacillus insect control protein gene as described in International Publication No. WO 99/31248, U.S. Pat. Nos. 5,689,052, 5,500,365 and 5,880,275. In another embodiment, the structural gene can confer tolerance to the herbicide glyphosate as conferred by genes including, but not limited to Agrobacterium strain CP4 glyphosate resistant EPSPS gene (aroA:CP4) as described in U.S. Pat. No. 5,633,435, or glyphosate oxidoreductase gene (GOX) as described in U.S. Pat. No. 5,463,175. Alternatively, the DNA coding sequences can affect these phenotypes by encoding a non-translatable RNA molecule that causes the targeted inhibition of expression of an endogenous gene, for example via antisense- or cosuppression-mediated mechanisms. The RNA could also be a catalytic RNA molecule (e.g., a ribozyme) engineered to cleave a desired endogenous mRNA product. See, e.g., Gibson and Shillito, MOL. BIOTECH., 7:125, 1997. Thus, any gene which produces a protein or mRNA which is necessary for a phenotype or morphology change of interest is useful for the practice of one or more embodiments.

Single-Gene Conversions

Single gene conversions of ‘PAN2020’, ‘HURV19PAN’, and/or ‘HURV2019CKH’ are included as embodiments of the present invention. The term single gene converted plant as used herein refers to those Cannabis plants which are developed by backcrossing, wherein in backcrossing essentially all of the desired morphological and physiological characteristics of a variety are recovered in addition to the single gene transferred into the variety via the backcrossing technique. Backcrossing methods can be used with one embodiment of the present application to improve or introduce a characteristic into the variety. The term “backcrossing” as used herein refers to the repeated crossing of a hybrid progeny back to the recurrent parent, e.g., backcrossing 1, 2, 3, 4, 5, 6, 7, 8, or more times to the recurrent parent. The parental Cannabis plant that contributes the gene for the desired characteristic is termed the nonrecurrent or donor parent. This terminology refers to the fact that the nonrecurrent parent is used one time in the backcross protocol and therefore does not recur. The parental Cannabis plant to which the gene or genes from the nonrecurrent parent are transferred is known as the recurrent parent as it is used for several rounds in the backcrossing protocol. In a typical backcross protocol, the original variety of interest (recurrent parent) is crossed to a second variety (nonrecurrent parent) that carries the single gene of interest to be transferred. The resulting progeny from this cross are then crossed again to the recurrent parent and the process is repeated until a Cannabis plant is obtained wherein essentially all of the desired morphological and physiological characteristics of the recurrent parent are recovered in the converted plant, in addition to the single transferred gene from the nonrecurrent parent.

The selection of a suitable recurrent parent is an important step for a successful backcrossing procedure. Often the goal of a backcross protocol is to alter or substitute a single trait or characteristic in the original variety. To accomplish this, a single gene of the recurrent variety is modified or substituted with the desired gene from the nonrecurrent parent, while retaining essentially all of the rest of the desired genetic, and therefore the desired physiological and morphological constitution of the original variety. The choice of the particular nonrecurrent parent will depend on the purpose of the backcross; a common purpose is to add some commercially important trait or traits to the plant. The exact backcrossing protocol will depend on the characteristic or trait being altered to determine an appropriate testing protocol. Although backcrossing methods are simplified when the characteristic being transferred is a dominant allele, a recessive allele may also be transferred. In this instance, it may be necessary to introduce a test of the progeny to determine if the desired characteristic has been successfully transferred.

Many single gene traits have been identified that are not regularly selected for in the development of a new variety but that can be improved by backcrossing techniques. These single gene traits are well-known in the art.

Backcross Conversions of ‘PAN2020’, ‘HURV19PAN’, and ‘HURV2019CKH’

A backcross conversion of ‘PAN2020’, ‘HURV19PAN’, or ‘HURV2019CKH’ occurs when DNA sequences are introduced through backcrossing with ‘PAN2020’, ‘HURV19PAN’, or ‘HURV2019CKH’, respectively, utilized as the recurrent parent. Both naturally occurring DNA sequences and transgenic DNA sequences may be introduced through backcrossing techniques. A backcross conversion may produce a plant with a trait or locus conversion in at least two or more backcrosses, including at least 2 crosses, at least 3 crosses, at least 4 crosses, at least 5 crosses, and the like. Molecular marker assisted breeding or selection may be utilized to reduce the number of backcrosses necessary to achieve the backcross conversion. It has been demonstrated in the art that a backcross conversion can be made in as few as two backcrosses.

The complexity of the backcross conversion method depends on the type of trait being transferred (e.g., single genes or closely linked genes as compared to unlinked genes), the level of expression of the trait, the type of inheritance (cytoplasmic or nuclear), and the types of parents included in the cross. It is understood by those of ordinary skill in the art that for single gene traits that are relatively easy to classify, the backcross method is effective and relatively easy to manage. Desired traits that may be transferred through backcross conversion include, but are not limited to, cannabinoid profile of high CBG with no THC and no CBD, sterility (nuclear and cytoplasmic), fertility restoration, drought tolerance, nitrogen utilization, Cannabis features, disease resistance (bacterial, fungal, or viral), insect resistance, and herbicide resistance. In addition, an introgression site itself, such as an FRT site, Lox site, or other site specific integration site, may be inserted by backcrossing and utilized for direct insertion of one or more genes of interest into a specific plant variety. In some embodiments, the number of loci that may be backcrossed into ‘PAN2020’, ‘HURV19PAN’, or ‘HURV2019CKH’ is at least 1, 2, 3, 4, or 5, and/or no more than 6, 5, 4, 3, or 2. A single locus may contain several transgenes, such as a transgene for disease resistance that, in the same expression vector, also contains a transgene for herbicide resistance. The gene for herbicide resistance may be used as a selectable marker and/or as a phenotypic trait. A single locus conversion of site specific integration system allows for the integration of multiple genes at the converted loci.

The backcross conversion may result from either the transfer of a dominant allele or a recessive allele. Selection of progeny containing the trait of interest may be accomplished by direct selection for a trait associated with a dominant allele. Transgenes or genes transferred via backcrossing typically function as a dominant single gene trait and are relatively easy to classify. Selection of progeny for a trait that is transferred via a recessive allele typically requires growing and selfing the first backcross generation to determine which plants carry the recessive alleles. Recessive traits may require additional progeny testing in successive backcross generations to determine the presence of the locus of interest. The last backcross generation may be selfed to give pure breeding progeny for the gene(s) being transferred, although a backcross conversion with a stably introgressed trait may also be maintained by further backcrossing to the recurrent parent with selection for the converted trait.

In addition, the above process and other similar processes described herein may be used to produce first generation progeny Cannabis seed by adding a step at the end of the process that comprises crossing ‘PAN2020’, ‘HURV19PAN’, or ‘HURV2019CKH’ with the introgressed trait or locus with a different plant and harvesting the resultant first generation progeny seed.

MOLECULAR TECHNIQUES USING ‘PAN2020’, ‘HURV19PAN’, and/or ‘HURV2019CKH’

Molecular biological techniques have allowed the isolation and characterization of genetic elements with specific functions. Traditional plant breeding has principally been the source of new germplasm; however, advances in molecular technologies have allowed breeders to provide varieties with novel and desired commercial attributes. Molecular techniques such as transformation are popular in breeding Cannabis plants and well-known in the art.

Breeding with Molecular Markers

Molecular markers may also be used during the breeding process for the selection of qualitative traits. For example, markers closely linked to alleles or markers containing sequences within the actual alleles of interest may be used to select plants that contain the alleles of interest during a backcrossing breeding program. The markers may also be used to select for the genome of the recurrent parent and against the genome of the donor parent. Using this procedure can minimize the amount of genome from the donor parent that remains in the selected plants. It can also be used to reduce the number of crosses back to the recurrent parent needed in a backcrossing program. The use of molecular markers in the selection process is often called genetic marker enhanced selection. Molecular markers may also be used to identify and exclude certain sources of germplasm as parental varieties or ancestors of a plant by providing a means of tracking genetic profiles through crosses. Molecular markers, which include, but are not limited to, markers identified through the use of techniques such as Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs), and Single Nucleotide Polymorphisms (SNPs), may be used in plant breeding methods utilizing ‘PAN2020’.

One use of molecular markers is Quantitative Trait Loci (QTL) mapping. QTL mapping is the use of markers, which are known to be closely linked to alleles that have measurable effects on a quantitative trait. Selection in the breeding process is based upon the accumulation of markers linked to the positive effecting alleles and/or the elimination of the markers linked to the negative effecting alleles from the plant's genome. See, e.g., Fletcher, et al., QTL analysis of root morphology, flowering time, and yield reveals trade-offs in response to drought in Brassica napus (2014) JOURNAL OF EXPERIMENTAL BIOLOGY. 66 (1): 245-256. QTL markers may also be used during the breeding process for the selection of qualitative traits. For example, markers closely linked to alleles or markers containing sequences within the actual alleles of interest can be used to select plants that contain the alleles of interest during a backcrossing breeding program.

Production of Double Haploids

The production of double haploids can also be used for the development of plants with a homozygous phenotype in the breeding program. For example, a Cannabis plant for which ‘PAN2020’, ‘HURV19PAN’, or ‘HURV2019CKH’ is a parent can be used to produce double haploid plants. Double haploids are produced by the doubling of a set of chromosomes (1N) from a heterozygous plant to produce a completely homozygous individual. This can be advantageous because the process omits the generations of selfing needed to obtain a homozygous plant from a heterozygous source.

Thus, an embodiment is a process for making a substantially homozygous Cannabis ‘PAN2020’ progeny plant by producing or obtaining a seed from the cross of ‘PAN2020’ and another Cannabis plant and applying double haploid methods to the F₁ seed or F₁ plant or to any successive filial generation.

In particular, an embodiment is a process of making seed retaining the molecular marker profile of ‘PAN2020’, such process comprising obtaining or producing F₁ seed for which ‘PAN2020’ is a parent, inducing doubled haploids to create progeny without the occurrence of meiotic segregation, obtaining the molecular marker profile of ‘PAN2020’, and selecting progeny that retain the molecular marker profile of ‘PAN2020’.

Thus, an embodiment is a process for making a substantially homozygous Cannabis ‘HURV19PAN’ progeny plant by producing or obtaining a seed from the cross of ‘HURV19PAN’ and another Cannabis plant and applying double haploid methods to the F₁ seed or F₁ plant or to any successive filial generation.

In particular, an embodiment is a process of making seed retaining the molecular marker profile of ‘HURV19PAN’, such process comprising obtaining or producing F₁ seed for which ‘HURV19PAN’ is a parent, inducing doubled haploids to create progeny without the occurrence of meiotic segregation, obtaining the molecular marker profile of ‘HURV19PAN’, and selecting progeny that retain the molecular marker profile of ‘HURV19PAN’.

Thus, an embodiment is a process for making a substantially homozygous Cannabis ‘HURV2019CKH’ progeny plant by producing or obtaining a seed from the cross of ‘HURV2019CKH’ and another Cannabis plant and applying double haploid methods to the F₁ seed or F₁ plant or to any successive filial generation.

In particular, an embodiment is a process of making seed retaining the molecular marker profile of ‘HURV2019CKH’, such process comprising obtaining or producing F₁ seed for which ‘HURV2019CKH’ is a parent, inducing doubled haploids to create progeny without the occurrence of meiotic segregation, obtaining the molecular marker profile of ‘HURV2019CKH’, and selecting progeny that retain the molecular marker profile of ‘HURV2019CKH’.

Signal Sequences for Targeting Proteins to Subcellular Compartments

Transport of a protein produced by a transgene to a sub cellular compartment, such as the chloroplast, vacuole, peroxisome, glyoxysome, cell wall, or mitochondrion, or for secretion into the apoplast, may be accomplished by means of operably linking a nucleotide sequence encoding a signal sequence (targeting sequence) typically to the 5′ and/or 3′ region of the transgene encoding the protein of interest. Signal sequences are well-known in the art. See, e.g., Becker, et al., PLANT MOL. BIOL., 20:49 (1992); Knox, et al., PLANT MOL. BIOL., 9:3-17 (1987); Lerner, et al., PLANT PHYSIOL., 91:124-129 (1989); Frontes, et al., PLANT CELL, 3:483-496 (1991); Matsuoka, et al., PROC. NATL. ACAD. SCI., 88:834 (1991); Gould, et al., J. CELL. BIOL., 108:1657 (1989); Creissen, et al., PLANT J., 2:129 (1991); Kalderon, et al., CELL, 39:499-509 (1984); and Steifel, et al., PLANT CELL, 2:785-793 (1990).

Gene Silencing

Techniques for gene silencing are well-known in the art, including, but not limited to, knock-outs (such as by insertion of a transposable element such as Mu) or other genetic elements such as a FRT, Lox, or other site specific integration sites; antisense technology; co-suppression; RNA interference; virus-induced gene silencing; target-RNA-specific ribozymes; hairpin structures; MicroRNA; ribozymes; oligonucleotide mediated targeted modification; Zn-finger targeted molecules; CRISPR/Cas9 system; and other methods or combinations of the above methods known to those of skill in the art. See, e.g., Sheehy, et al., PNAS USA, 85:8805-8809 (1988); U.S. Pat. Nos. 5,107,065; 5,453,566; 5,759,829; Jorgensen, TRENDS BIOTECH., 8(12):340-344 (1990); Flavell, PNAS USA, 91:3490-3496 (1994); Neuhuber, et al., MOL. GEN. GENET., 244:230-241 (1994); Napoli, et al., PLANT CELL, 2:279-289 (1990); U.S. Pat. No. 5,034,323; Sharp, GENES DEV., 13:139-141 (1999); Zamore, et al., CELL, 101:25-33 (2000); Montgomery, et al., PNAS USA, 95:15502-15507 (1998); Burton, et al., PLANT CELL, 12:691-705 (2000); Baulcombe, CURR. OP. PLANT BIO., 2:109-113 (1999); Haseloff, et al., NATURE, 334:585-591 (1988); Smith, et al., NATURE, 407:319-320 (2000); U.S. Pat. Nos. 6,423,885; 7,138,565; 6,753,139; 7,713,715; Aukerman & Sakai, PLANT CELL, 15:2730-2741 (2003); Steinecke, et al., EMBO J., 11:1525 (1992); Perriman, et al., ANTISENSE RES. DEV., 3:253 (1993); U.S. Pat. Nos. 6,528,700; 6,911,575; 7,151,201; 6,453,242; 6,785,613; 7,177,766; 7,788,044; International Publication No. WO2014/068346; Martinelli, et al., Proposal of a Genome Editing System for Genetic Resistance to Tomato Spotted Wilt Virus 2014 AMERICAN JOURNAL OF APPLIED SCIENCES; Noman, et al., CRISPR-Cas9: Tool for Qualitative and Quantitative Plant Genome Editing, November 2016 FRONTIERS IN PLANT SCIENCE Vol. 7; and Zhang et al., Exploiting the CRISPR/Cas9 System for Targeted Genome Mutagenesis in Petunia February 2016 SCIENCE REPORTS Volume 6.

Tissue Culture

Further reproduction of the variety can occur by tissue culture and regeneration. Tissue culture (cell culture) of various tissues of plants and regeneration of plants therefrom are well-known and widely published. See, e.g., Rego, et al., CROP BREEDING AND APPLIED TECHNOLOGY. 1(3): 283-300 (2001); Komatsuda, et al., CROP SCI., 31:333-337 (1991); Stephens, et al., THEOR. APPL. GENET., 82:633-635 (1991); Komatsuda, et al., PLANT CELL, TISSUE AND ORGAN CULTURE, 28:103-113 (1992); Dhir, et al., PLANT CELL REPORTS, 11:285-289 (1992); and Shetty, et al., PLANT SCIENCE, 81:245-251 (1992). Thus, another embodiment is to provide cells which upon growth and differentiation produce Cannabis plants having the physiological and morphological characteristics of ‘PAN2020’, ‘HURV19PAN’, or ‘HURV2019CKH’, described in the present application.

Regeneration refers to the development of a plant from tissue culture or cell culture. Exemplary types of tissue cultures or cell cultures are protoplasts, calli, plant clumps, and plant cells that can generate tissue culture that are intact in plants or parts of plants, such as pollen, ovules, embryos, protoplasts, meristematic cells, callus, pollen, leaves, ovules, anthers, cotyledons, hypocotyl, pistils, roots, root tips, flowers, seeds, petiole, shoot, or stems, and the like. Means for preparing and maintaining plant tissue culture and plant cell cultures are well-known in the art.

While a number of exemplary aspects and embodiments have been disclosed above, those of skill in the art will recognize certain modifications, permutations, additions, and sub-combinations thereof. The foregoing discussion of the embodiments has been presented for purposes of illustration and description. The foregoing is not intended to limit the embodiments to the form or forms disclosed herein.

Moreover, though the description of the embodiments has included description of one or more embodiments and certain variations and modifications, other variations and modifications are within the scope of the embodiments (e.g., may be within the skill and knowledge of those in the art, after understanding the present disclosure). It is intended to obtain rights which include alternative embodiments to the extent permitted, including alternate, interchangeable and/or equivalent structures, functions, ranges or acts to those claimed, whether or not such alternate, interchangeable and/or equivalent structures, functions, ranges or acts are disclosed herein, and without intending to publicly dedicate any patentable subject matter.

### Deposit Information

A deposit of each of ‘PAN2020’ and ‘HURV2019CKH’ plant tissue disclosed herein will be made in accordance with all of the requirements of 37 C.F.R. §§ 1.801-1.809. Moreover, seeds of ‘HURV19PAN’ disclosed herein will be made in accordance with all of the requirements of 37 C.F.R. §§ 1.801-1.809.

We claim:

1. A tissue or cell culture of regenerable cells produced from a Cannabis plant of variety selected from the group consisting of ‘PAN2020’, ‘HURV19PAN’, and ‘HURV2019CKH’ wherein a representative sample of plant tissue of said variety or seed producing said variety is to be deposited.

2. The tissue or cell culture of claim 1, comprising tissues or cells from a plant part selected form the group consisting of leaves, pollen, embryos, cotyledons, ovules, protoplasts, callus, pollen, seeds, petiole, hypocotyl, meristematic cells, roots, root tips, pistils, anthers, flowers, and stems.

3. A Cannabis plant regenerated from the tissue or cell culture of claim 1.

4. A method of developing a Cannabis plant variety having the physiological and morphological characteristics of a Cannabis plant of variety selected from the group consisting of ‘PAN2020’, ‘HURV19PAN’, and ‘HURV2019CKH’, wherein a representative sample of plant tissue of said variety or seed producing said variety is to be deposited, said method comprising genotyping a Cannabis plant of said variety wherein said genotyping comprises obtaining a sample of nucleic acids from said plant and detecting in said nucleic acids a plurality of polymorphisms, and using said identified polymorphisms for marker assisted selection in a breeding program.

5. A method for developing a Cannabis plant variety, comprising one or more of:
a) identifying and selecting a spontaneous mutation of a Cannabis plant of variety selected from the group consisting of ‘PAN2020’, ‘HURV19PAN’, and ‘HURV2019CKH’, or a part thereof, and cultivating said selected spontaneous mutation plant or plant part;
b) introducing a mutation into the genome of a plant of variety selected from the group consisting of ‘PAN2020’, ‘HURV19PAN’, and ‘HURV2019CKH’, or a part thereof, and cultivating said mutated plant or plant part; or
c) transforming a Cannabis plant of variety selected from the group consisting of ‘PAN2020’, ‘HURV19PAN’, and ‘HURV2019CKH’, with a transgene;
wherein a representative sample of plant tissue of said variety or seed producing said variety is to be deposited.

6. A Cannabis plant produced by cultivating said selected spontaneous mutation plant or plant part of claim 5.

7. A Cannabis plant produced by cultivating said mutated plant or plant part of claim 5.

8. The method of claim 5, wherein said mutation is introduced using a method selected from the group consisting of temperature, long-term seed storage, tissue culture conditions, ionizing radiation, chemical mutagens, targeting induced local lesions in genomes, zinc finger nuclease mediated mutagenesis, CRISPR/Cas9, meganucleases, and gene editing.

9. The method of claim 5, wherein said transgene confers resistance to an herbicide, insecticide, or disease.

10. An herbicide, insecticide, or disease resistant plant produced by the method of claim 9.

11. A method of producing an F1 seed or embryo, wherein the method comprises crossing a Cannabis plant of variety selected from the group consisting of ‘PAN2020’, ‘HURV19PAN’, and ‘HURV2019CKH’, with a second plant and harvesting the resultant F1 seed or embryo,
wherein a representative sample of plant tissue of said variety or seed producing said variety is to be deposited.

12. The method of claim 11, wherein said second plant comprises another plant of variety selected from the group consisting of ‘PAN2020’, ‘HURV19PAN’, and ‘HURV2019CKH’.

13. The method of claim 11, wherein said second plant is a plant of a different variety.

14. A Cannabis plant produced by cultivating the harvested F1 seed or embryo of claim 11.

15. A Cannabis plant, or plant part, tissue, or cell thereof, which produces a female inflorescence, said inflorescence comprising:
(a) a cannabigerol (CBG) content that is at least 10%,
(b) a tetrahydrocannabinol (THC) content of less than 0.3%, and
(c) a cannabidol (CBD) content of less than 0.3%; and
wherein said CBG content, said THC content, and said CBD content is based on dry weight of the inflorescence.

16. The Cannabis plant, or plant part, tissue, or cell thereof of claim 15, wherein said THC content is less than 0.1% and said CBD is less than 0.1%.

17. The Cannabis plant, or plant part, tissue, or cell thereof of claim 15, wherein said THC content is less than 0.05% and said CBD is less than 0.05%.

18. The Cannabis plant, or plant part, tissue, or cell thereof of claim 15, wherein said CGB content is greater than 15%.

19. The Cannabis plant, or plant part, tissue, or cell thereof of claim 15, wherein said THC content is absent and said CBD is absent.

20. A method of breeding said Cannabis plant of claim 15, comprising:
(i) making a cross between a first Cannabis plant and a second Cannabis plant to produce an F1 seed,
(ii) harvesting the resulting seed,
(iii) growing said seed, and
(iv) selected a Cannabis plant with a desired phenotype; and
wherein the resulting selected Cannabis plant has at least 10% CBG content, less than 0.3% THC content, and less than 0.3% CBD content.

21. An extract from the Cannabis plant or a plant part, tissue, or cell thereof of claim 15, or from an asexual clone of said Cannabis plant.

22. The extract of claim 21, wherein said extract is selected from the group consisting of kief, hashish, bubble hash, solvent reduced oils, sludges, e-juice, and tinctures.

23. An edible product comprising Cannabis tissue from the Cannabis plant, or a plant part, tissue, or cell thereof of claim 15, or from an asexual clone of said Cannabis plant.

24. An edible product comprising the extract of claim 21.
